(19) United States
(12) Patent Application Publication
(10) Pub. No.: US 2012/0276139 A1
(43) Pub. Date: Nov. 1, 2012

(54) USE OF NEWCASTLE DISEASE VIRUS-BASED VECTOR FOR INDUCING AN IMMUNE RESPONSE IN MAMMALS

(75) Inventors: Robertus Jacobus Maria Moormann, Dronten (NL); Jeroen Alexander Kortekaas, Zwolle (NL); Adrianus Franciscus Gerardus Antonis, Garderen (NL); Bernardus Petrus Hubertus Peeters, Lelystad (NL); Aldo Dekker, Eemnes (NL)

(73) Assignee: Stichting Dienst Landbouwkundig Onderzoek, Wageningen (NL)

(21) Appl. No.: 13/510,109

(22) PCT Filed: Nov. 16, 2010
(86) PCT No.: PCT/NL2010/050763

§ 371 Date: Jul. 18, 2012

(30) Foreign Application Priority Data
Nov. 16, 2009 (EP) 09176152.8

(51) Int. Cl. A61K 39/00; A61P 37/04; A61K 39/15; A61P 31/20; A61K 39/12; A61P 31/14

(52) U.S. Cl. 424/199.1

(57) ABSTRACT

The invention relates to methods of stimulating an immune response against an antigenic protein in a mammalian subject. More specifically, the invention relates to routes of administration of a hybrid Newcastle Disease Virus-vector (NDV-vector) for eliciting an immune response against an antigenic protein that is encoded by the hybrid NDV-vector.

### FIELD

The present invention relates to the field of vaccines, more specifically, to methods for immunizing mammals with an avian virus-based vector. The invention especially relates to the routes of administration of a Newcastle Disease Virus-based vector to mammals which provide protection against infectious disease.

Newcastle disease virus (NDV) is a member of the Avulavirus genus of the Paramyxoviridae family (Fauquet et al., 2005. Virus Taxonomy: Eighth Report of the International Committee on Taxonomy of Viruses, Academic Press). NDV is exclusively pathogenic for birds and highly pathogenic strains can cause severe economic losses in the poultry industry (Alexander, 1997. In: Calnek B W, Barnes H J, Beard C W, L R McDougal, Saif Y M (eds) Diseases of poultry, 10th edition. Iowa State University Press, Ames, pp 541-569). Vaccination against NDV using highly attenuated strains such as strain “LaSota” is common practice. The availability of an NDV reverse genetics system has opened up ways to use NDV as a vaccine vector (Zhao and Peeters, 2003. J Gen Virol 84: 781-8). Besides the widely explored possibilities of using recombinant NDV strains as vaccine vectors for application in poultry (Huang et al., 2004. J Virol 78: 10054-63; Park et al., 2006, Proc Natl Acad Sci USA 103: 8203-8; Veits et al., 2006. Proc Natl Acad Sci USA 103: 8197-202), there are several advantages of using NDV as a vaccine vector for mammals as well (Bukreyev et al., 2006. J Virol 80: 10293-306). The most important advantages result from the fact that mammals are not natural hosts for NDV. This minimizes the chance of vaccination failure due to pre-existing immunity in the field. Furthermore, there is generally little or no virus spread in the inoculated mammal (Bukreyev and Collins, 2008. Curr Opin Mol Ther 10: 46-55; Bukreyev et al., 2005. J Virol 79: 13275-84; DiNapoli et al., 2007. Proc Natl Acad Sci USA 104: 9788-93), rendering the use of NDV in mammals inherently safe. Despite the restricted replication of NDV in mammals, foreign genes can be expressed efficiently from the NDV genome and several promising NDV-based vector vaccines for use in mammals have been developed already (Bukreyev and Collins, 2008. Curr Opin Mol Ther 10: 46-55; Bukreyev et al., 2005. J Virol 79: 13275-84; DiNapoli et al., 2007. Proc Natl Acad Sci USA 104: 9788-93; Dinapoli, et al., 2009. Vaccine 27: 1530-9; DiNapoli et al., 2007. J Virol 81: 11560-8).

It was recently demonstrated that inoculation of calves with NDV via a combined intranasal/intratracheal route, resulted in a systemic antibody response against NDV vector proteins without causing any clinical signs (Subbiah et al., 2008. Arch Virol 153: 1197-200). It has also been reported that parental administration of NDV failed to elicit immune responses and that effective immunization requires delivery through the respiratory tract (DiNapoli et al., 2009. Vaccine 27: 1530-1539).

The present invention provides a method of stimulating an immune response against an antigenic protein in a mammalian subject comprising administering a composition comprising a hybrid Newcastle Disease Virus-vector (NDV-vector) comprising a nucleotide sequence encoding the antigenic protein to the subject through parenteral administration.

NDV-based vaccines are generally administered via the respiratory tract. When using lentogenic strains for the vaccination against respiratory diseases of poultry, this is a logical choice, since this inoculation route ensures optimal cleavage of the F protein by trypsin-like proteases of the respiratory tract and thereby ensures optimal vaccine efficacy. For application in mammals this application route is also generally selected (Bukreyev et al., 2005. J Virol 79: 13275-84; DiNapoli et al., 2007. Proc Natl Acad Sci USA 104: 9788-93; DiNapoli et al., 2007. J Virol 81: 11560-8).

The inventors surprisingly recognized that, in contrast to current concepts, administration of an NDV-vector to a mammal via a parenteral route is much more potent in inducing a systemic antibody response against both the vector and the antigenic protein when compared with administration via the respiratory route.

The term parenteral refers to a route of administration which is selected from intravenous, intra-arterial, intramuscular, subcutaneous, intradermal, and intraperitoneal administration. Preferred routes for administering of the NDV-vector are intradermal, subcutaneous, and, most preferred, intramuscular administration. Further preferred is a combined subcutaneous/intradermal route. The term parenteral does not include nasal and/or intratracheal administration, for example through inhalation or the use of nose-sprays.

Parenteral routes, preferably a subcutaneous/intradermal route and/or an intramuscular route, for administration of a hybrid NDV-vector are fast, generally between 10 seconds and 5 minutes and normally result in 100% bioavailability of the hybrid NDV-vector transducing an antigenic protein.

The term subcutaneous/intradermal route refers to a combination of subcutaneous and intradermal injection, which can be performed simultaneously or consecutively. Subcutaneous injections pierce the epidermal and dermal layers of the skin and deliver the drug into the loose subcutaneous tissue. The site is usually the loose skin between the shoulder blades or the triceps area of the foreleg or forearm. Alternatively, the ventral abdomen is commonly used. The intradermal route aims at delivering the hybrid NDV-vector in the space between the outer epidermis and the underlying dermis. Intramuscular injections are preferably given deep into skeletal muscles, typically into the gluteal, deltoid, rectus femoris, or vastus lateralis muscles of a mammal. The choice of the injection site is based on a desire to minimize the chance of the needle hitting a nerve or blood vessel.

Without being bound by theory, the vast microcirculatory blood and lymphatic plexuses in/below the dermis and the enhanced blood flow in muscles may provide an improved absorption profile for administered substances. In addition, the temperature at the site of subcutaneous/intradermal administration and/or intramuscular route administration may have a positive effect on the immune response. Especially the elevated temperature of muscles, compared to the temperature in, for example, the nasal or intra-tracheal area, might have a positive effect on replication of NDV, which natural host is a bird with a body temperature of about 41° C.

Parenteral administration can be performed by, for example, injection or infusion. Further preferred is the use of a needle-free device that drive liquid medication through a nozzle orifice, creating a narrow stream under high pressure that penetrates skin for intradermal, subcutaneous, or intramuscular administration of the composition comprising an NDV-based vector to a mammalian subject.

A composition according to the invention preferably further comprises an adjuvant. Adjuvant substances are used to stimulate immunogenicity. Examples of commonly used immunological adjuvants are aluminum salts, immunostimulating complexes (ISCOMS), non-ionic block polymers or copolymers, cytokines (like IL-1, IL-2, IL-7, etc.), saponins, monophosphoryl lipid A (MLA), muramyl dipeptides, vitamin E, polyacrylate resins, and oil emulsions. Preferably, the adjuvant is a sulfohpopolysaccharide, such as the SLP/S/W adjuvant described in Hilgers et al. Vaccine 1994 12:653-660. A further preferred adjuvant is provided by a triterpene, such as squalene, and derivative and modifications therefore.

The induced effector immune response may either be of a humoral nature, i.e. an antibody response, or of a cellular nature, i.e. a cytotoxic T-cell response, or the effector response may be a mixture of both. In this respect, it is important to note that NDV is a potent inducer of α and β interferons (Blach-Olszewska, 1970. Arch Immunol Ther Exp (Warsz) 18(4): 418-41; Brehm and Kirchner, 1986. J Interferon Res 6: 21-8), which are known to enhance antigen presentation through the MHC class I pathway (Biron, 2001. Immunity 14: 661-4; Honda et al. 2003. Proc Natl Acad Sci USA. 100: 10872-10877; Le Bon et al. 2003. Nature Immunology 4: 1009-1015; Santini et al. 2000. The Journal of Experimental Medicine 191: 1777-1788). Accordingly, the inherently high adjuvant activity of NDV mediated by CD8+ T cell responses has recently been demonstrated (Martinez-Sobrido et al. 2006. J Vir 80: 1130-1139).

Both cellular and humoral immune responses require help from T helper lymphocytes. Adjuvants that cause inflammation or induce pro-inflammatory cytokines will induce a Type-1 T helper response involving production of InterLeukin-12 (IL-12), IL-2 and interferon-gamma. Non-inflammatory adjuvants are more likely to induce a Type-2 helper response involving production of the cytokines IL-4, IL-5 and IL-10. Further examples of an adjuvant that can be used in a method of the invention are chemically or genetically detoxified bacterial toxins, such as the cholera toxin or lymphotoxin from Escherichia coli, saponins such as QuilA and QS21, muramyl di- or tripeptides and derivatives, glycosylceramide, such as, for example, α-galactosylceramide, liposomes based on, for example, phosphatidylcholine, dioleylphosphatidylethanolamine, 1-methyl-4-(cis-9-dioleyl)methyl-pyridinium-chlorid, N-[1-(2,3-dioleoyloxy)propyl]-N,N,Ntrimethylammonium methylsulfate and/or mixtures thereof, CpG oligonucleotides, and any combination thereof. Preferably, the adjuvant is a sulfohpopolysaccharide, such as the SLP/S/W adjuvant described in Hilgers et al. Vaccine 1994 12:653-660.

In addition, a composition according to the invention may further comprise a stabilizing agent selected from the group consisting of non-reducing sugars including, for example, sucrose, trehalose, stachyose, or raffinose, polysaccharides such as, for example, dextran, soluble starch and dextrin, reducing sugars such as, for example, monosaccharides such as apiose, arabinose, lyxose, ribose, xylose, digitoxose, fucose, quercitol, quinovose, rhamnose, allose, altrose, fructose, galactose, glucose, gulose, hamamelose, idose, mannose and tagatose; and disaccharides such as, for example, primeverose, vicianose, rutinose, scillabiose, cellobiose, gentiobiose, lactose, lactulose, maltose, melibiose, sophorose, and turanose, and cyclodextrins such as, for example, alpha-cyclodextrin, beta-cyclodextrin, gamma-cyclodextrin, glucosyl-alpha-cyclodextrin, maltosyl-alpha-cyclodextrin, glucosyl-beta-cyclodextrin, maltosyl-beta-cyclodextrin, hydroxypropyl beta-cyclodextrin, 2-hydroxypropyl-beta-cyclodextrin, 2-hydroxypropyl-gamma-cyclodextrin, hydroxyethyl-beta-cyclodextrin, methyl-beta-cyclodextrin, sulfobutylether-alpha-cyclodextrin, sulfobutylether-beta-cyclodextrin, and sulfobutylether-gamma-cyclodextrin.

In a preferred method according to the invention, the parenteral administration is repeated. According to this embodiment, said parenteral administration is performed two times, three times, or four times. If the parenteral administration is performed two or more times, each of the two or more parenteral administrations is independently selected from intravenous, intra-arterial, intramuscular, subcutaneous, intradermal, and intraperitoneal administration, more preferred from intradermal, subcutaneous, intravenous and intramuscular administration. In addition, each of the two or more parenteral administrations may independently comprise one or more adjuvants selected from interleukin, cholera toxin or lymphotoxin from Escherichia coli, saponin such as QuilA and QS21, muramyl di- or tripeptides and derivatives, glycosylceramide, such as, for example, α-galactosylceramide, liposomes based on, for example, phosphatidylcholine, dioleylphosphatidylethanolamine, 1-methyl-4-(cis-9-dioleyl)methyl-pyridinium-chlorid, N-[1-(2,3-dioleoyloxy)propyl]-N,N,N, trimethylammonium methylsulfate and/or mixtures thereof, CpG oligonucleotides, and any combination thereof.

The immune response that is stimulated by a method of the invention preferably protects the subject against an infectious disease. Said infectious disease can be mediated by a bacterium, such as for example, salmonellosis, campylobacteriosis, anthrax, botulism, brucellosis, tuberculosis, leptospirosis, plague, Q-fever, shigellosis and tularaemia, diseases mediated by a parasite such as, for example, cysticercosis, taeniasis, echinococcosis, hydatidosis, toxoplasmosis and trematodosis, a rickettsial disease such as, for example, bovine anaplasmosis, and a virus such as, for example, rabies virus, influenza virus, Crimean-Congo haemorrhagic fever virus, Ebola virus or Rift Valley fever virus.

In a preferred embodiment, the stimulated immune response after parenteral administration of a composition comprising a hybrid NDV-vector protects the subject against an infectious disease selected from, for example, salmonellosis, campylobacteriosis, anthrax, botulism, brucellosis, leptospirosis, plague, shigellosis, tularaemia, cysticercosis, taeniasis, echinococcosis, hydatidosis, rabies, anthrax, Japanese encephalitis, Marburg haemorrhagic fever, Q Fever, sheep pox, goat pox, equine encephalomyelitis, African swine fever, classical swine fever, contagious bovine pleuropneumonia, foot and mouth disease, bluetongue, peste des petits ruminants, rinderpest, stomatitis, enteritis, acquired immune deficiency syndrome, Rift Valley fever, African trypanosomiasis, influenza, Buruli ulcer disease, cholera, Crimean-Congo haemorrhagic fever, dengue, ebola, hepatitis, Cache Valley fever, Lassa fever, legionellosis, leprosy, malaria, meningitis, plague, poliomyelitis, smallpox, tuberculosis and yellow fever, African horsesickness, equine encephalosis, Eastern equine encephalitis, Western equine encephalitis, SARS, West Nile encephalitis, Nipah virus disease, hantavirus pulmonary syndrome, hantavirus hemorrhagic fever with renal syndrome, Hendra virus infections.

The invention further provides a method according to the invention, wherein the stimulated immune response protects the subject against a subsequent infection with a transmitter of an infectious disease. Said transmitter is preferably selected from Adenovirus, African horsesickness virus, African swine fever, Arbovirus, Bluetongue virus, Border disease virus, Borna virus, Bovine viral diarrhoe virus, Bunyavirus, Cache valley fever virus, Chikungunya virus, Chrysomya bezziana, Classical swine fever, Crimean-congo hemorrhagic fever virus, Cochliomyia hominivorax, Coronavirus, Cytomegalovirus, Dengue virus, Eastern equine encephalitis virus, Ebola virus, Equine encephalomyelitis virus, Equine encephalosis virus, Foot and mouth disease virus, Goat pox virus, Hantavirus, Hendra virus, Hepatitis A virus, Hepatitis B virus, Hepatitis C virus, Hepatitis E virus, Herpes simplex virus, Highly pathogenic avian influenza virus, Human immunodeficiency virus, human parainfluenza virus, Influenza virus, Japanese encephalitis virus, Kaposi's sarcoma-associated herpesvirus, Lassa virus, Lujo virus, Marburg virus, Marsilia virus, Measles virus, Monkeypox virus, Mumps virus, Nipah virus, Papillomavirus, Papova virus, Peste des petits ruminants, Polio virus, Polyomavirus, Rabies virus, Respiratory syncytial virus, Rhinovirus, Rift Valley fever, Rinderpest, Rotavirus, Rubella virus, Sandfly fever Naples virus, Sandfly fever Sicilian virus, SARS coronavirus, Sheep pox virus, Simian immunodeficiency virus, Smallpox virus, St. Louis encephalitis virus, Toscana virus, Varicella-zoster virus, West Nile virus, Western equine encephalitis virus, Yellow fever virus, Bacillus anthracis, Bacillus anthracis, Bordetella pertussis, Brucella spp., Campylobacter jujuni, Chlamydia trachomatis, Clostridium botulinum, Coxiella burnettii, Francisella tularensis, Group B streptococcus, Legionella pneumophila, Leptospira spp., Mycobacterium leprae, Mycobacterium tuberculosis, Mycobacterium ulcerans, Neisseria meningitidis, Salmonella, Shigella spp., Trypanosoma cruzi, Vibrio cholerae, Yersinia pestis, Mycoplasma mycoides, Plasmodium malariae, Plasmodium ovale, Plasmodium ssp., Plasmodium vivax, Taenia solium, Taenia spp., and Trypanosoma brucei.

It will be clear to a skilled person that a stimulated immune response protects a subject against a subsequent infection with a transmitter of an infectious disease if the antigenic protein that is used in a method according to the invention is a protein that is expressed by the transmitter of an infectious disease, or an immunologically-active part or derivative of a protein that is expressed by the transmitter of an infectious disease. For example, a stimulated immune response protects a subject against a subsequent challenge with Rift Valley fever virus (RVFV), if the antigenic protein that is used in a method according to the invention is a protein that is expressed by RVFV, or an immunologically-active part or derivative of a protein that is expressed by RVFV. Method for determining whether a protein, or a part or derivative of a protein, is immunologically active are known to the person skilled in the art, including algorithms that predict the immunogenicity of a protein such as an algorithm of Parker and an algorithm of Rammensee, as disclosed in Provenzano et al. 2004. Blood 104: Abstract 2862) and including the injection of the purified protein, or a part or derivative of the protein in a suitable animal and determining whether the protein, or a part or derivative of a protein is capable of stimulating antibodies against the protein, or a part or derivative of a protein.

The term immunologically-active part indicates a part of a protein that is able to induce a cellular and/or humoral immune response against the protein in a mammalian subject. The term immunologically-active derivative indicates a protein or part of a protein that is modified, for example by addition, deletion or alteration of one or more amino acids and which is able to induce a cellular and/or humoral immune response against the protein in a mammalian subject. It is preferred that an immunologically-active derivative has a sequence identity of more than 70% compared to the protein that is expressed by the transmitter of an infectious disease, more preferred more than 80%, more preferred more than 90%, more preferred more than 95%, more preferred more than 99%, most preferred 100%, as based on the amino acid sequence of the protein or protein parts. Said immunologically-active derivative is, for example, a protein that comprises a signal peptide for secretion out of the cell in which it is produced, a protein that comprises a sequence that provides a trans-membrane such as a type I, II or III targeting domain, or a protein in which a protease cleavage site has been altered to enhance the half-life of the protein.

The term “sequence identity” refers to the percentage of identical matches between a protein and the above-mentioned amino acid sequence.

In a preferred embodiment, the stimulated immune response in a method according to the invention protects the subject against infection by Rift Valley fever virus (RVFV), Bluetongue virus and/or Crimean-congo hemorrhagic fever virus.

RVFV is a mosquito-borne, enveloped phlebovirus of the Bunyaviridae family that can cause severe disease in ruminants and humans. Case fatality is extremely high in young animals and the fatality rate for foetuses in pregnant livestock can approach 100% (Bird et al., 2009. J Am Vet Med Assoc 234: 883-93; Coetzer, 1977. Onderstepoort J Vet Res 44: 205-11; Coetzer, J. A., 1982. Onderstepoort J Vet Res 49: 11-7). The disease in humans is generally mild, although a small percentage of individuals suffer from serious sequelae, such as fulminant hepatitis, encephalitis, ocular damage or hemorrhagic fever (Al-Hazmi et al., 2003. Clin Infect Dis 36: 245-52; McIntosh et al., 1980. S Afr Med J 58: 803-6). The virus is endemic in Africa and the Arabian peninsula, where it causes recurrent outbreaks of large socio-economic impact.

A preferred antigenic protein is selected from the group comprising RNA-dependent RNA polymerase, NSm protein, Gn glycoprotein, Gc glycoprotein, the N protein, and the NS protein, or an immunologically-active part or immunologically-active derivative thereof. A most preferred antigenic protein is provided by the Gn glycoprotein. Other preferred antigenic proteins are the Gc glycoprotein and the N protein. Preferably, the antigenic protein is RVFV glycoprotein Gn or Gc, or virus-like particles produced by expression of both Gn and Gc from the NDV genome

Further preferred is the expression of at least two antigenic proteins that are selected from the group comprising RNA-dependent RNA polymerase, NSm protein, Gn glycoprotein, Gc glycoprotein, the N protein, and the NS protein, or an immunologically-active part or immunologically-active derivative thereof. Said at least two antigenic proteins can be independently expressed on the hybrid NDV-vector. Preferred examples are Gn glycoprotein and N protein, Gc glycoprotein and N protein, or a combination of Gn/Gc and N protein. Further preferred is the expression of a pre-protein which, for example comprises the NSm protein, Gn, and/or Gc or an immunologically-active part or immunologically-active derivative thereof. Protease recognition sequences may be provided in between the proteins that mediate cleavage of the pre-protein into the individual proteins or immunologically-active parts or immunologically-active derivatives thereof.

Bluetongue is a non-contagious viral disease of both domestic and wild ruminants. The double stranded RNA virus, termed Bluetongue virus (BTV), is endemic in some areas with cattle and wild ruminants serving as reservoirs for the virus. Preferred antigenic proteins are selected from VP2 and/or V5.

Crimean-congo hemorrhagic fever virus is a member of the genus Nairovirus, family Bunyaviridae. Preferred antigenic proteins are selected from the mature virus glycoproteins, Gn and Gc (previously referred to as G2 and G1).

The invention therefore provides a hybrid NDV-vector comprising a nucleotide sequence encoding the antigenic protein for use as a vaccine to protect a mammalian subject after parenteral administration of the vaccine to the mammalian subject against an infectious disease.

The term “vaccine” as used herein refers to a pharmaceutical composition comprising at least one immunologically active antigenic protein that induces an immunological response in a mammal and possibly, but not necessarily, one or more additional components that enhance the immunological activity of the active component. A vaccine may additionally comprise further components typical to pharmaceutical compositions.

An antigenic protein that is expressed by a hybrid NDV-vector according to the invention provides one or more sub-cellular components derived from a pathogen of interest. As is known to the skilled person, this subunit vaccine antigenic protein preferably is presented to the immune system such that strong humoral immunity and strong cell-mediated immunity are induced. The use of one or more adjuvant substances, including interleukins, may stimulate immunogenicity, as is known to a person skilled in the art.

The vector that is used in a method of the invention is a hybrid NDV-vector. NDV is a member of the genus Avulavirus in the family Paramyxoviridae and contains a nonsegmented single-stranded RNA genome of negative polarity containing six major genes in the order of 3′-NP-P-M-F-HN-L-5′. A system based on cotransfection of a plasmid expressing full-length antigenomic RNA together with three other plasmids encoding viral NP, P, and L proteins under control of the phage T7 RNA polymerase promoter, which resulted in the recovery of recombinant viruses, was first developed for rabies virus (Schnell et al., 1994. EMBO J 13: 4195-4203) and subsequently for NDV (Peeters et al., 1999. J Virol 73: 5001-5009).

NDV causes an economically important disease in all species of birds worldwide. Besides the widely explored possibilities of using recombinant NDV strains as vaccine vectors for application in poultry (Huang et al., 2004. J Virol 78: 10054-63; Park et al., 2006. Proc Natl Acad Sci 103: 8203-8; Veits et al., 2006. Proc Natl Acad Sci 103: 8197-202), there are several advantages of using NDV as a vaccine vector for mammals as well (Bukreyev et al., 2006. J Virol 80: 10293-306). The most important advantages result from the fact that mammals are not natural hosts for NDV. This minimizes the chance of vaccination failure due to pre-existing immunity in the field. Furthermore, there is generally little or no virus spread in the inoculated mammal (Bukreyev and Collins, 2008. Curr Opin Mol Ther 10: 46-55; Bukreyev et al., 2005. J Virol 79: 13275-84; DiNapoli et al., 2007. Proc Natl Acad Sci 104: 9788-93), rendering the use of NDV in mammals inherently safe. Despite the restricted replication of NDV in mammals, foreign genes can be expressed efficiently from the NDV genome and several promising NDV-based vector vaccines for use in mammals have been developed already (Bukreyev and Collins, 2008. Curr Opin Mol Ther 10: 46-55; Bukreyev et al., 2005. J Virol 79: 13275-84; DiNapoli et al., 2007. Proc Natl Acad Sci 104: 9788-93; Dinapoli et al., 2009. Vaccine 27: 1530-9; DiNapoli et al., 2007. J Virol 81: 11560-8).

NDV strains have been classified as pathogenic (mesogenic or velogenic) or non-pathogenic (lentogenic) to poultry. Differences between lentogenic strains and pathogenic strains are present in the HN protein and the F protein. The HN protein, which is responsible for virus attachment to receptors, varies in length due to differences in the sizes of the ORFs. An HN protein precursor of 616 aa has been found in lentogenic but not in pathogenic NDV strains. The F protein, which mediates virus-cell fusion, requires proteolytic activation at an internal cleavage site, whose amino acid composition determines cleavability by various proteases. Thus, the length of the HN protein, in combination with the F protein cleavage site, are important factors for the virulence of NDV strains.

A preferred NDV-vector for use in a method according to the invention is a lentogenic vector. Examples of lentogenic NDV strains are LaSota, Ulster, F, Queensland, MC, Sz, and Hitchner B1. Examples of recombinant vector that are based on lentogenic NDV strains are NDFL (Peeters et al., 1999. J Virol 73: 5001-5009) which is based on LaSota; pflNDV-1 (Roemer-Oberdoerfer et al., 1999. J Gen Vir 80: 2987-2995) which is based on LaSota clone 30, KBNP-C4152R2L (Cho et al., 2008. Clin and Vaccine Immunol 15: 1572-1579) which is based on LaSota, and pNDV/B1 (Nakaya et al., 2001. J Virol 75: 11868-11873) which is based on the Hitchner B1 strain. A further preferred NDV-vector is NDFL or similar infectious clone of NDV strain LaSota.

A preferred NDV vector is NDFL. NDFLtag is an infectious clone with the fusion cleavage site sequence of the F-protein mutated to the virulent motif. NDFL and NDFLtag have been described (Peeters et al., 1999. J Virol 73: 5001-5009).

A hybrid NDV vector for use in a method of the invention comprises a heterologous gene that encodes an antigenic protein or immunologically-active part or derivative of the antigenic protein. Said heterologous gene is present in an expression cassette that mediates expression of the antigenic protein from the heterologous gene in cells that comprise the hybrid NDV vector. RNA synthesis can be initiated from a NDV-promoter. Plasmids encoding the antigenic protein are available in the art, and can be obtained flanked by linker sequences for convenient manipulation, if desired. In a preferred embodiment, the antigenic protein is encoded in the delivery NDV-virion as a preproprotein, preferably positioned in between the P and M-protein of NDV. In a preferred embodiment, the reading frame of the antigenic protein is flanked by NDV transcription start and stop sequences. Alternatively, for expression of a large antigenic protein or multiple antigenic proteins, a two segmented NDV system can be generated wherein each of the two segments is flanked by authentic NDV 3′ and 5′ noncoding termini allowing for efficient production of the virus (Gao et al., 2008. J Vir 82: 2692-2698).

In a preferred embodiment, the nucleotide sequence of the heterologous gene that encodes the antigenic protein or immunologically-active part or derivative of the antigenic protein is optimized for expression in a mammalian cell. A codon-optimized heterologous gene can achieve higher levels of expression compared to a non-optimized gene. The sequence of the heterologous gene can further be amended to modify the secondary and/or tertiary structure, and/or to modify cis-acting elements in the DNA or RNA-expression product that may modulate transcription and/or translation of the heterologous gene.

A mammalian subject for applying a method according to the invention is preferably selected from larger animals. Said larger animals include pets such as dog and cat; ungulates including pig, horse and ruminants such as sheep, cow; and goat; and primates, including human. Most preferred mammalian subjects are humans, ruminants, horses, and pets.

### FIGURE LEGENDS

FIG. 1. Expression of BTV-8 VP2 or VP5 in recombinant NDV infected cells. Shown are IPMAs of QM-5 cells infected with 10⁴- to 10⁵-fold diluted allantoic fluid containing NDFL_RV-VP2 (panels a-c) or NDFL-VP5 (panels d-f) and stained using a mAb specific for NDV F protein (panels a,d), a guinea pig BTV-8 immune serum (panels b,e) or a rabbit antiserum against a peptide of VP5 (panels c,f). Scale bars, 100 μm.

FIG. 2. Antibody responses against NDV elicited in sheep that were immunized with various NDV viruses by different immunization routes. In the first experiment, groups of two sheep were immunized with rNDV-VP2 (a) or rNDV-VP5 (b) by either the i.m. or the i.n./i.t. route. In another experiment groups of four sheep were immunized with live (c) or inactivated (d) wildtype NDV LaSota virus by the i.m., s.c./i.d. or i.n./i.t. route. Immunizations were repeated at 21 days post primary immunization. Serum samples were then analyzed for presence of NDV specific antibodies by ELISA. Geometric mean titres and standard deviations are shown. The Y-axis starts at 2.7 10 log titre, corresponding to the lowest serum dilution analyzed (500-fold).

FIG. 3. Construction of the recombinant NDFL-Gn strain. (A) Nucleotide sequence of the cassette in plasmid pGEM-PM-cassette. The Gn gene was introduced between the LguI sites. (B) Construction of NDFL-Gn. Transcription start and stop boxes are indicated in white and black, respectively.

FIG. 4. Detection of the Gn glycoprotein on NDFL-Gn infected BHK-21 cells. Cells were infected with NDV strain NDFL or NDFL-Gn, fixed with 4% paraformaldehyde at day 4 post infection and used for immunostaining with a RVFV polyclonal antiserum and subsequently with FITC-conjugated rabbit anti-sheep polyclonal antibodies.

FIG. 5. Western blot showing the presence of Gn in allantoic fluid. Allantoic fluid (Mock) or allantoic fluid containing NDFL or NDFL-Gn was placed on top of a 20% sucrose solution and centrifuged at 80 000×g for 2 h. Previously harvested culture supernatant of S2 cells containing RVFV VLPs was included as a reference. The proteins present in the resulting pellets were separated on NuPAGE gels and detected on Western blots using a rabbit antiserum raised against a peptide derived from the Gn protein. The positions of the Gn monomer and an oligomer containing Gn are indicated by arrowheads. The position of molecular weight standard proteins are indicated to the left.

FIG. 6. NDV specific IgG response elicited by NDFL or NDFL-Gn after intranasal (interrupted lines) or intramuscular (solid lines) inoculation of calves as determined by indirect ELISA. The results depicted are averages (n=3, ±S.D.).

FIG. 7. Construction of the recombinant NDFL-GnGc strain. (A) Nucleotide sequence of the cassette in plasmid pGEM-PM-cassette. The GnGc gene was introduced between the LguI sites. (B) Construction of NDFL-GnGc. Transcription start and stop boxes are depicted in white and black, respectively.

FIG. 8. Western blots showing the presence of Gn and Gc in allantoic fluid. Allantoic fluid (Mock) or allantoic fluids containing NDFL-Gn or NDFL-GnGc were placed on top of a 20% sucrose solution and centrifuged at 80.000×g for 2 h. Previously harvested culture supernatant of Drosophila S2 cells containing RVFV VLPs (de Boer et al., submitted for publication) was included as a reference. The proteins present in the resulting pellets were separated on NuPAGE gels and detected on Western blots using rabbit polyclonal antibodies raised against a Gn (panel A) or Gc (panel B)-derived peptide. The positions of the Gn (˜54 kDa) and Gc (˜59 kDa) [Gerrard et al. 2007. Virology 357: 124-338] monomers are indicated by arrowheads. The positions of molecular weight standard proteins are indicated to the left.

FIG. 9. Survival curve. Mice were either left untreated or vaccinated on days 0 and 21 with NDFL or NDFL-GnGc and subsequently challenged on day 42 with virulent RVFV strain M35/74.

### EXAMPLES

### Example 1

### Materials and Methods

Cells and viruses. Quail muscle (QM-5) cells grown in Ford Dodge QT35 medium (Invitrogen, Carlsbad, Calif.) containing 5% fetal calf serum (FCS) were used for virus titration and recovery of recombinant NDV virus. We used a reverse genetics system for production of recombinant viruses based on the lentogenic LaSota strain published previously. The nonrecombinant NDV LaSota strain was originally derived from the ATCC (VR-699) and passaged three times in the allantoic cavity of 9- to 11-day-old embryonated chicken eggs prior to use in animal experiments. NDV viruses were stored at −70° C. in allantoic fluid. Formalin-inactivated virus was generated by addition of formalin to a final concentration of 0.1% (v/v) and 2 days incubation at 4° C.

A control NDV virus producing a Rift Valley fever virus antigen was produced in a similar manner as described in the next section (J. Kortekaas, manuscript in preparation).

Generation of recombinant NDV viruses. Synthetic genes encoding VP2 or VP5 of BTV-8 Net2006/04, codon-optimized for expression in human cells, were obtained from Genscript Corporation (Piscataway, USA). The genes were inserted into a plasmid named pGEM-PM-cassette (kindly provided by Olav de Leeuw, CVI-WUR, Lelystad, The Netherlands). The pGEM-PM-cassette plasmid contains the sequence that is located between unique ApaI and NotI sites in the pNDFL plasmid [Peeters et al., 1999. J Vir 73: 5001-9] as well as newly introduced transcription start and stop boxes and two LguI sites that can be used for insertion of foreign genes. The sequence between the ApaI and NotI of plasmid pNDFL was exchanged for the corresponding region of plasmid pGEM-PM-cassette-VP5 plasmid. This resulted in insertion of the VP5 gene between the NDFL P and M genes. The resulting pNDFL-VP5 plasmid was used for transfection of QM-5 cells to recover NDFL-VP5 as described previously [Peeters et al., 1999. J Vir 73: 5001-9]. This virus was readily recovered from embryonated eggs.

The nucleotide sequence of NDFL differs from the consensus sequence of the LaSota strain (GenBank accession number AF077761.1). Although we were successful in rescuing a recombinant NDV virus that contains the VP5 gene (1581 bps), we chose to repair these mutations before attempting rescue of NDFL strains with larger inserted foreign genes, such as VP2 (2886 bps). The nucleotide differences result in four mutations: F protein, R189 (Q); HN protein D393 (N); L protein, Q97 (E) and K191 (R) (consensus between parentheses). In addition, the LaSota consensus sequence that was used to construct NDFL contains an asparagine (N) at position 369 in the L protein. Since all other NDV strains contain an isoleucine (I) at this position, including other LaSota isolates (AAW30681.1, CAB51327.1), we chose to change the corresponding N369 codon of NDFL to I. The resulting plasmid, named pNDFL_RV, was used to insert the VP2 gene, resulting in pNDFL_RV-VP2.

The codon optimized open reading frame used for VP2 is as follows:

ATGGAAGAACTGGCTATTCCAATCTATACAAATGTGTTCCCTGCTGAG
CTTCTGGATGGCTATGATTATATTATAGATGTGTCTTCCAGGGTGGAG
GAGGAAGGTGATGAGCCAGTCAAGAGACATGATGTGACAGAGATCCC
AAGGAACTCTATGTTTGATATTAAGGATGAACATATCAGAGATGCTAT
TATATATAAGCCAGTCAACAATGATGGCTATGTGCTGCCCAGGGTGCT
GGATATCACTCTGAAGGCTTTTGATGATAGAAAGAGGGTGGTCCTGA
ATGATGGCCATTCTGAGTTCCATACAAAAACAAACTGGGTGCAGTGG
ATGATTGATGATGCTATGGATGTTCAGCCCCTGAAGGTGGATATTGCA
CACACAAGGTCAAGGATATCTCATGCCCTCTTTAACTGCACAGTCAGA
CTCCACAGCAAGAAGGCTGACACAGCCTCTTACCATGTGGAGCCTGT
GGAAATTGAAAGTTGGGGATGTAATCACACATGGCTTAGTAGGATTC
ACCACCTGGTTAATGTGGAACTGTTTCACTGCTCTCAGGAAGCTGCAT
ATACACTGAAGCCAACCTACAAGATCATCAGCAATGCAGAGAGAGCC
TCAACATCTGATTCCTTCAATGGTACTATGATTGAGCTTGGCAGGAAC
CACCAAATCCAAATGGGTGATCAGGGATACCAGAAACTGAAAGAGGG
CTTGTGCAAGTGAGGATTGAAGGCAAAACCCCACTGGTGATCCAGGA
AGAATTACTGCATTGAACAAAATTAGAGAGCAATGGATTGCAAGAAAT
TTTGACCAGAGAGAGATAAAAGTCCTGGACCTTTGTAGGCTGTTGTCA
ACCATTGGAAGAAAAATGTGCAACACAGAGGAGGAGCCAAAAAATGA
GGCTGATCTGAGTGTGAAGTTTCAGATGGAGCTTGATGAGATTTTCA
GGCCAGGTAATAATGAGAGAACAAACATCATGGGAGGAGGAGTGCAT
AGAAAAAATGAGGATAGGTTCTATGTGCTGATTATGATTGCTGCCTCT
GACACCAATAAGGGCAGGATCTGGTGGTCCAACCCTTATCCTTGTCT
GAGAGGAGCCCTGATAGCTGCAGAAGTGCAGCTGGGTGATGTCTACA
ACCTCCTTAGGAATTGGTTTCAGTGGTCAGTCAGGCCAACTTATGTCC
TTATGATAGGAACAGGGAGTCAGACAAGTACATCTACAGCAGGATCA
ACCTGTTTGACAGTACCTTGAGGCCAGGTGATAAGATAGTGCACTGG
GAGTACAAACTGCTGAATGAGGTGAGAGAGGTTAGCATTAACAAGGG
GAATGAGTGTGACCTGTTCCCAGAGGATGAGGAGTTCACAACAAAAT
TCCATGAGGCCAGGTATACAGAAATGAAGAATCAGATCATACAGAGT
GATGGAATCAAAGAGACTTTAAGATGCATAAGATCTTGGAGGATGGA
GCTAATGTGTTGACCATAGACTTTGAGAAAGATGCTCATATAGGGACT
GGTTCAGCTCTGAGTCTGCCAGACTATTACAACAAGTGGATCATTGCC
CCTATGTTCAATGCCAAGCTCAGAATTACAGAAGTGGTGATTGGGACT
GCACACACAGATGACCCTGCAGTGGGTAGGAGTGCCAAGGCATTCAC
ACATGACCCATTTGATTTGCAAAGGTATTGCCTGGCCAGATATTATGA
TGTCAGGCCTGGAATGATGGGTAGAGCCCTGTCCAAACAGCAGAACA
TGTCATCCATGACTGATAAACTGTCCAAACAGGAGGACTATGCTGGC
ATTGTTAGTAGGAGACTTGAATACAAAGAAAGGGAGAATAGATGTCT
GACTGAGACTGCACAGTATGTCTTTGAAAAGACTTGCCTTTATGTGCT
GGAGCTGCTGTCAAGGCATACCATGCCTTCAGAGGACAGTGAGGTCA
CCTTTGAACATCCCACCATTGACCCATCAGTGGACATAGAGACCTGGA
AGATCATAGATGTCTCCCAGCTTATTATATTTGTTTTTGATTATCTGTT
TGAGAACAGAAAGATAGTCAGAGACACTACAGAGGCAAGGTGGACCC
TGTTTAAGATCAGGAGTGAAGTGGGCAGGGCAAGGAATGATGCCATT
GAAATGACCTTTCCCAGGTTTGGCAGGATGCTCAGAAATGCATCCCA
GGCCAAAATCAACCAGGACATTGCCTGTCTGAACTTTCTGCCCCTTCT
GTTCATCATTGGTGACAATATCAGCTATGCCCATAGGCAGTGGTCT
ATTCCAGTTCTTCTGTATGCTCATGATATCAGGATTATCCCCCTGGAA
GTTGGGGCTTATAACAACAGATTTGGCCTGACCTCATACCTTGAGTAC
ATGGCATTCTTCCCAAGTTATGCAACAAGAGTGGCCAAAATTGATGAG
AGCATCAAAGAGTGTGCCATTGCTATGGCAGAGTTCTACATGAACACT
GATATCCACTCTGGATCTGTTATGAGCAATGTGATCACAACAAAGAGA
TTGCTGTATGAGACTTACCTGGCATCCCTGTGTGGAGGTTATTCTGAT
GGGCTCTTGTGGTACTTGCCAATCACCCATCCTAGCAAGTGCCTGGT
GGCCTTTGAAGTTGCTGATGATGTTGTGCCCCTGAGTGTCAGGAGAG
AGAGGATCCTGTCAAGGTTTCCTCTCTCATCTAGGCATGTGAAGGGA
ATAGCTCTGATCAGTGTGGACAGGAACCAGAAGGTGTCTGTCCAGAC
AGAGGGAATTGTGACCCACAGACTGTGCAAAAAGAACTTGCTTAAGT
ATGTGTGTGATGTGATTCTGTTCAAGTTCTCTGGACATGTGTTTGGAA
ATGATGAGATGTTGACCAAGCTGCTTAATGTTTAA

The codon optimized open reading frame used for VP5 is as follows:

ATGGGCAAGATCATCAAGTCACTGAGCAGGTTTGGGAAGAAAGTGGG
CAATGCTTTGACTAGCAACACAGCTAAGAAGATCTATTCTACCATTGG
AAAAGCTGCAGAGAGGTTTGCAGAGTCAGAAATAGGCAGTGCTGCTA
TTGATGGTCTGGTGCAGGGTTCTGTCCACTCCTTGATGACAGGGGAG
TCTTATGGGGAATCAGTTAAACAGGCTGTGCTCCTTAATGTCATGGGC
TCTGGAGAGGAACTGCCTGACCCACTTTCTCCTGGGGAAAGGGGGAT
GCAGACCAAAATTAGGGAACTGGAGGATGAGCAGAGAAATGAACTGA
TTAGGCTGAAGTACAATGACAAAATCAAGCAGAAGTTTGGCAAGGAG
TTGGAGGAGGTGTATGAGTTCATGAATGGGGTGGCCAAGCAGGAAGA
AGATGAGGAAAAACACTATGATGTGCTCAAGAAGGCAGTGAACAGTT
ATGACAAGATCCTGACTGAAGAGGAAAAGCAAATGAGGATCCTGGCT
ACAGCCCTGCAGAAGGAGGTGAAGGAAAGAACTGGGACAGAGGCTG
TGATGGTGAAAGAGTATAGAAATAAGATTGATGCCCTGAAGGAGGCT
ATTGAGGTGGAAAGAGATGGTATGCAGGAAGAAGCCATCCAGGAGAT
TGCTGGCATGACAGCAGATGTGCTGGAGGCTGCTAGTGAGGAGGTGC
CCCTCATTGGTGCTGGGATGGCCACAGCTGTTGCCACAGGCAGAGCC
ATTGAGGGTGCCTATAAGCTGAAAAAAGTGATCAATGCTTTGAGTGG
CATTGATCTCACACATCTCAGAACTCCCAAAATTGAACCTACCATTGTG
AGCACAGTGCTTGATCACAAATTCAAGGACATTCCTGATGAGATGCTGG
CAGTGTCTGTTCTGTCCAAAAACAGAGCCATTGAAGAGAACCACAAAGA
GATCATCCACCTTAAGAATGAAATCCTCCCAAGGTTTAAGAAGGCAATG
GATGAGGAGAAGGAGATCTGTGGTATTGAGGACAAGAAGATACATCCAA
AGGTTATGATGAAGTTTAAAATTCCTAGAACCCAGCAGCCTCAGATTCA
CATCTACTCTGCACCTTGGGATTCTGATGATGTGTTTTTCTTCCATTGC
ATCAGTCACCACCATGCTAATGAGTCTTTCTTCATTGGATTTGATCTGG
GAATTGATCTGGTCCATTATGAAGACCTCACAGCACACTGGCATGCACT
GGGGGCAGCTCAGGCTGCTGTGGGTAGATCCCTCAATGAAGTGTACAAG
GAGTTCCTGAACTTGGCTATCAATAATACTTACTCCAGTCAAATGCATG
CCAGGAGGATGATTAGATCAAAAACAGTGCATCCAATTTATTTGGGTAG
TCTCCACTATGACATCTCCTTCAGCACACTTAGGAGTAATGCACAGAGG
ATTGTGTATGATGAAGAGTTGCAGATGCACATCCTGAGAGGGCCTTTGC
ACTTCCAGAGGAGAGCCATTCTGGGGGCCATTAAGCATGGAGTGAAGAT
TCTGGGCACTGAGGTGGATATCCCTCTCTTCCTGAGGAATGCTTAG

Virus passaging and sequencing. NDFL_RV-VP2 and NDFL-VP5 viruses were rescued by inoculation of transfection supernatant into 9- to 11-day-old embryonated hen's eggs. The recovery of infectious virus was confirmed by standard haemagglutination assays. To establish genetic stability, NDFL_RV-VP2 and NDFL-VP5 were passaged five times in embryonated eggs. For sequence analysis, viral RNA was isolated (QIAamp MinElute Virus Spin Kit, Qiagen, Hilden, Germany) and the fragments flanked by LguI sites encoding the BTV-8 VP2 or VP5 proteins were amplified by reverse transcriptase-polymerase chain reaction (RT-PCR). Sequence analysis was performed using VP2 or VP5 gene specific primers, the BigDye Terminator v1.1 Cycle Sequencing Kit and an automated ABI3130 DNA sequencer (Applied Biosystems, Nieuwerkerk a/d IJssel, The Netherlands).

Virus titration and neutralization test. NDFL_RV-VP2 and NDFL-VP5 titres were determined by limiting dilution on monolayers of QM-5 cells and examination of NDV infected cells using an immunoperoxidase monolayer assay specific for the F-protein (see next section) at 3-4 days post infection. The tissue culture 50% infectious dose (TCID₅₀) was calculated according to the method of Spearmann and Karber [Karber, 1931. Arch Exp Path Pharmak 162: 480-83; Spearman, 1908. Br J Psychol 2: 227-42]. The nonrecombinant LaSota strain was titrated in a similar manner but using DF-1 cells and the Reed and Muench method [Reed and Muench, 1932. Am J Hyg 27: 493-97] for calculation of endpoint titers. BTV-8 neutralizing antibody titers in sheep serum samples were assessed by microtitre virus neutralization (VN) test using a constant amount of 100 TCID₅₀ of BTV8/Neth/2007 that was preincubated with serial twofold serum dilutions prior to infection of baby hamster kidney (BHK)-21 cells.

Immunoperoxidase monolayer assay. Monolayers of QM-5 or DF-1 cells infected with recombinant NDV viruses were fixed with paraformaldehyde (4% w/v in PBS) and subsequently washed with PBS. For detection of NDV, an F-protein-specific mouse monoclonal antibody (mAb 8E12A8C3) was used as the primary antibody. For detection of BTV-8 VP2 we used a polyclonal anti-BTV-8 serum that was obtained by intramuscular infection of an SPF guinea pig with 10⁷ TCID₅₀ BTV8/Neth/2007. For detection of VP5 we used a rabbit antiserum directed against a keyhole limpet hemocyanin conjugated peptide (ERDGMQEEAIQEIAGMTADVLEAASEEVPLIGAGMATAC; N-terminal acetylation) derived from a previously published conserved region of VP5 (Wade-Evans et al., 1988, Virus Research 11, 227-240) but containing an additional C-terminal cysteine for conjugation purposes. This antiserum was affinity purified using this same (unconjugated) peptide (Genscript Corporation). Antisera were diluted in His buffer (0.5M NaCl/1% Tween-80/0.1% NaN₃) and 4% horse serum. After incubation at 37° C. for 1 h, the plates were washed three times with PBS-T. Peroxidase-conjugated rabbit antibodies directed against either mouse or guinea pig immunoglobulins or swine antibodies directed against rabbit immunoglobulins (all from Dako, Glostrup, Denmark) were used as the secondary antibodies. Peroxidase activity was detected using 3-amino-9-ethyl-carbazole (Sigma, St. Louis, USA) as the substrate.

Animal experiments. Conventional sheep were used in immunization experiments. Animal experiments were performed under the supervision of the Animal Experimental Committee and were performed according to The Dutch Law on Animal Experiments.

Immunization with NDFL_RV-VP2 and NDFL-VP5. Sheep were divided into four groups of two animals. Each group received either NDFL_RV-VP2 or rNDFL-VP5 virus by either a combined intranasal/intratracheal (i.n./i.t.) route or the intramuscular (i.m.) route. NDV virus in allantoic fluid was concentrated 100-fold using centrifugal concentration devices with 100 kDa MWCO and was subsequently diluted about a 100-fold into PBS to 10⁷ TCID50 per ml. This material was administered via the i.n./i.t. route or i.m. route in a volume of 1 ml. Each group received identical booster immunizations at 21 days post primary immunization. Sera were collected weekly for a period of seven weeks.

Immunization with wildtype, non-recombinant NDV virus. Sheep were divided into seven groups of four animals each (Table 1). Three groups received NDV strain LaSota that was diluted 80-fold from allantoic fluid in PBS to 10⁷ TCID₅₀ per ml. This material was injected by either a combined i.n./i.t. route, a combined subcutaneous/intradermal (s.c./i.d.) route or the i.m. route, in a volume of 2, 1 or 2 ml, respectively, resulting in the doses indicated in Table 1. A further three groups received formalin-inactivated NDV virus by these three immunization routes. Control group seven received allantoic fluid of eggs that were not infected with NDV. Each group received booster immunizations at day 21 post primary immunization. Due to a human error, all animals of group 2 received a double volume (4 ml) of NDV virus at day 0, but the correct dose at day 21. Sera were collected weekly for a period of seven weeks.

TABLE 1
Administration of wildtype NDV LaSota to sheep.

| | | | | Dose |
|---|---|---|---|---|
| | Virus | | | (10log |
| Group | administration | Type of virus | Application Route | TCID₅₀) |
| 1 | Yes | Live | i.n./i.t. | 7.3 |
| 2 | Yes | Inactivated | i.n./i.t. | 7.3^{a} |
| 3 | Yes | Live | s.c./i.d. | 7.0 |
| 4 | Yes | Inactivated | s.c./i.d. | 7.0 |
| 5 | Yes | Live | i.m. | 7.3 |
| 6 | Yes | Inactivated | i.m. | 7.3 |
| 7 | No | NA^{b} | i.n./i.t. | NA |

^{a}All animals in this group received a double dose (7.6 10log TCID₅₀) at day 0, but a dose of 7.3 10log TCID₅₀ at day 21 post immunization. Note that titers reported for inactivated NDV refer to titers prior to formalin inactivation.
^{b}NA, not applicable.

NDV ELISA. Sera were examined for antibodies against NDV using plates coated with NDV obtained from a commercial ELISA for analysis of chicken sera (FlockChek Newcastle Disease Antibody Test Kit, IDEXX Laboratories, Hoofddorp, The Netherlands). Plates were blocked using ELISA-buffer (10% skimmed milk; 10% bovine serum albumin; 1% Tergitol NP-9; 0.05% Tween-80; 0.5 M NaCl; 2.7 mM KCl; 2.8 mM KH₂PO₄; 8.1 mM Na₂HPO₄; pH 7.4) and then incubated with 500-fold diluted sheep sera and further serial twofold dilutions in ELISA-buffer. Bound antibody was then detected with peroxidase-conjugated rabbit anti-sheep immunoglobulin G antibody (Abcam, Cambridge, UK) diluted into conjugate buffer (PBS containing 5% FBS; 2% NaCl; and 0.05% Tween-80) and staining with 3,3′,5,5′ tetramethylbenzidine. Using 4-parameter curve fitting we then interpolated the serum dilution resulting in an extinction at 450 nm of 0.2 above the background extinction observed without sheep serum.

### Results

Generation of recombinant NDV viruses encoding BTV VP2 or VP5. Genes encoding the VP2 or VP5 proteins of BTV-8 Net2006/04 were introduced as an additional transcription unit flanked by NDV-specific gene-start and gene-end signal sequences between the P and M genes of recombinant NDV strain LaSota (i.e. pNDFL). These inserts were designed such that the resulting viruses would comply with the rule of six [Peeters et al., 1999. J Vir 73: 5001-9]. Infectious NDFL_RV-VP2 and NDFL-VP5 viruses were produced by transfection of QM-5 cells and further propagated on embryonated eggs. Allantoic fluids of the second egg passage were used for virus characterization and animal experiments.

Characterization of recombinant NDV strains. The identity of the isolated NDFL_RV-VP2 and NDFL-VP5 viruses was confirmed by sequence analysis of the inserted genes. Both viruses yielded titers of about 10⁷ TCID₅₀/ml in embryonated eggs. To determine the stability of the inserted genes in the NDV genome, both viruses were passaged five times in embryonated eggs. Subsequent sequence analysis confirmed the integrity of the inserted genes of both viruses.

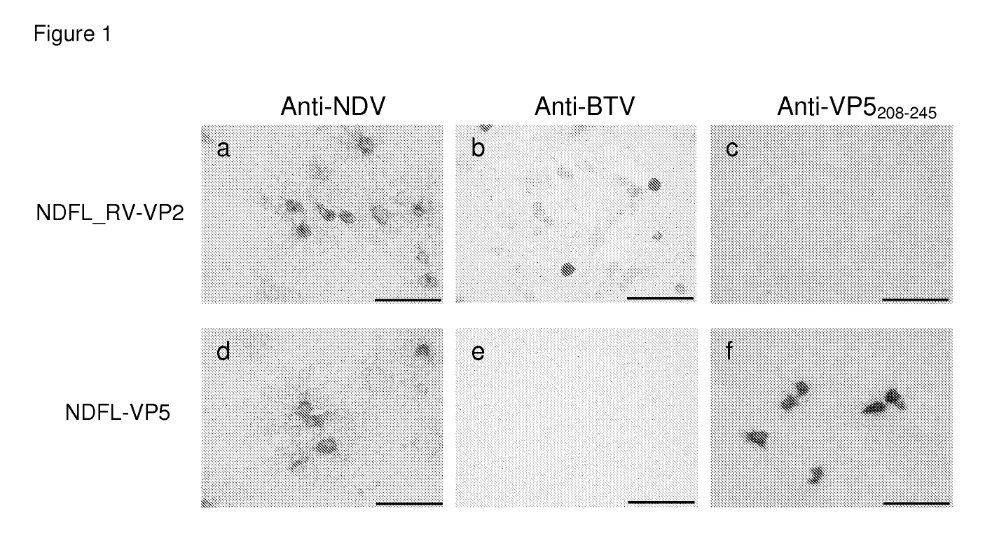

Expression of the VP2 protein of NDFL_RV-VP2 was demonstrated by IPMA on QM-5 cells using a polyclonal guinea pig serum directed against BTV8, that reacts positive on NDFL_RV-VP2 infected cells (FIG. 1b) but not on NDFL-VP5 infected cells (FIG. 1e). Similarly, VP5 expression could be demonstrated using an antiserum directed against a VP5 peptide that reacted positively with NDFL-VP5 infected QM-5 cells (FIG. 1f) but not with NDFL_RV-VP2 infected cells (FIG. 1c). As a control we demonstrated the presence of NDV virus using a mAb specific for NDV F protein (FIG. 1a,d).

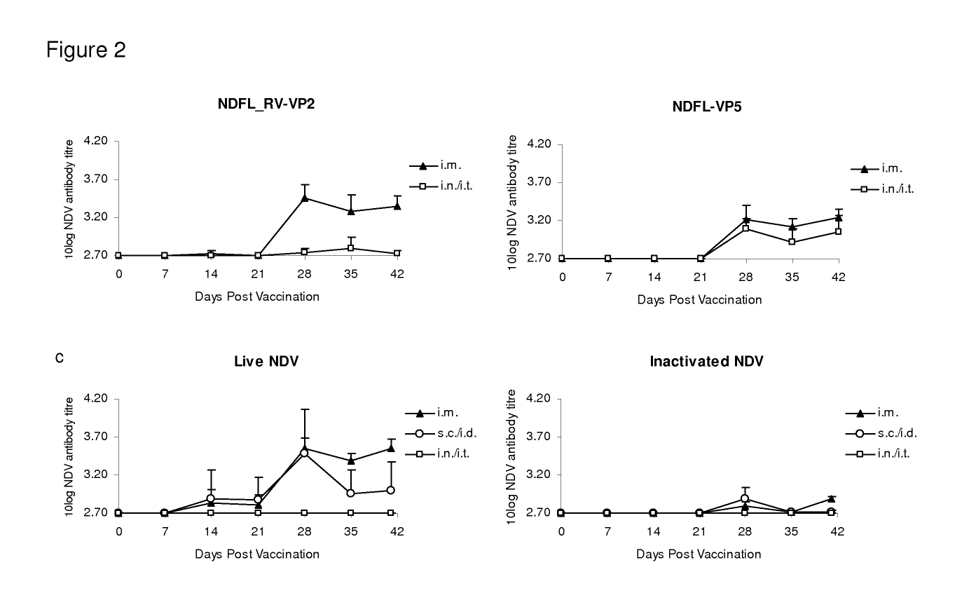

Immunogenicity of recombinant NDV strains. In a first animal experiment the immunogenicity of NDFL_RV-VP2 and NDFL-VP5 administered by either the i.n./i.t. or i.m. route was assessed. In both cases we could not detect an antibody response against the VP2 or VP5 proteins by either virus neutralization test, IPMA using BTV-8/2007/Neth-infected BHK-21 cells or by ELISA using plates coated with BTV-8 virus purified by sucrose density gradients (results not shown). As a control we determined the antibody response against NDV by ELISA (FIGS. 2a and b). Good antibody responses against NDV were detected as early as 28 days p.i., corresponding to 1 week after the booster immunization. This indicates that the absence of detectable immunogenicity of the inserted VP2 or VP5 genes was not due to inefficient NDV replication. Surprisingly, the immunogenicity of both recombinant viruses was higher when these were administered via the i.m. route when compared to the combined i.n./i.t route.

Determination of the optimal administration route of NDV-based vector vaccines. We determined the immunogenicity of NDV virus administered by the i.m., i.n./i.t. and s.c./i.d. routes, using four animals per group instead of two and using wildtype, nonrecombinant NDV strain LaSota to exclude that the observed effect was due to the presence of foreign genes. Furthermore, we used both live and inactivated NDV. A control group that did not receive NDV was always negative in NDV ELISAs (results not shown). Using live NDV we again observed superior immunogenicity when the virus was administered via the i.m. route. Immunogenicity of NDV administered via a combined s.c./i.d. route was of comparable efficacy, whereas NDV administered via the combined i.n./i.t. route resulted in lower antibody responses (FIG. 2c). Immunization with inactivated NDV resulted in far lower NDV antibody titers as compared to immunization with live NDV (FIGS. 2c and d), indicating that virus replication is necessary for optimal immunogenicity.

### Example 2

### Materials and Methods

Cells, plasmids and viruses. Quail muscle (QM-5) cells were grown in Ford Dodge QT35 medium (Invitrogen, Breda, The Netherlands) containing 5% fetal calf serum (FCS) and 1% antibiotic/antimycotic (Invitrogen). BHK-21 cells were grown in GMEM containing 4% tryptose phosphate broth (Invitrogen) and 10% FCS.

The cDNA clone of NDV strain LaSota, named NDFL and the helper plasmids pCIneo-NP, pCIneo-P and pCIneo-L, have been described previously (Peeters et al., 1999). Plasmid pCAGGS-GnGc contains a codon-optimized GnGc gene of strain M35/74 under chicken-actin promoter control.

The fowlpox recombinant virus fpEFLT7pol (hereafter called FPV-T7) (Britton et al., 1996) was provided by Olav de Leeuw (Central Veterinary Institute of Wageningen UR [CVI-WUR], Lelystad, The Netherlands). RVFV strain M35/74 was kindly provided by Prof. dr. Janusz Paweska (National Institute for Communicable Diseases [NICD], Johannesburg, South Africa) and Dr. Christiaan Potgieter (Agricultural Research Council-Onderstepoort Veterinary Institute [ARC-OVI], Onderstepoort, South Africa).

Construction of full-length recombinant cDNAs. The sequence of the M genome segment of RVFV strain M35/74 was kindly provided by Dr. Christiaan Potgieter (ARC-OVI). The sequence is as follows:

ATGGCAGGGATTGCAATGACAGTCCTTCCAGCCTTAGCAGTTTTTGCT
TTGGCACCTGTTGTTTTTGCTGAAGACCCTCATCTCAGAAACAGACCA
GGGAAGGGGCACAACTACATTGACGGGATGACTCAGGAGGACGCCAC
ATGCAAACCTGTGACATATGCTGGGGCTTGTAGCAGTTTTGATGTCTT
GCTCGAAAAGGGAAAATTCCCCCTCTTCCAGTCGTATGCCCATCACAG
AACCCTACTAGAAGCAGTTCACGACACCATCATTGCAAAGGCTGATCC
ACCTAGCTGTGACCTTCAGAGTGCTCATGGGAATCCCTGCATGAAGG
AGAAACTCGTGATGAAGACACACTGTCCAAATGACTACCAGTCAGCT
CATTACCTCAACAATGACGGGAAAATGGCTTCAGTCAAGTGCCCTCCT
AAATATGAGCTCACTGAGGACTGCAATTTTTGCAGGCAGATGACAGG
TGCTAGCTTGAAGAAGGGGTCTTATCCTCTTCAGGACTTATTTTGTCA
GTCAAGTGAGGATGATGGATCAAAATTAAAAACAAAAATGAAAGGGG
TCTGCGAAGTGGGGGTTCAAGCACTCAAAAAGTGTGATGGCCAACTC
AGCACTGCACATGAGGTTGTGCCCTTTGCAGTATTTAAGAACTCAAAG
AAGGTTTATCTTGATAAGCTTGACCTCAAGACTGAGGAAAATCTGTTG
CCAGACTCATTTGTCTGCTTCGAGCATAAGGGACAGTATAAAGGAAC
AATGGACTCTGGTCAGACCAAGAGGGAGCTCAAAAGCTTTGATATCT
CTCAGTGCCCCAAGATTGGAGGACATGGTAGCAAGAAGTGCACTGGG
GACGCAGCTTTTTGCTCTGCTTATGAGTGCACTGCTCAATACGCCAAT
GCTTATTGTTCACATGCTAATGGGTCAGGAGTTGTACAGATACAAGTA
TCCGGGGTCTGGAAGAAGCCTTTGTGTGTCGGGTATGAGAGGGTGGT
TGTGAAGAGAGAACTCTCTGCTAAGCCCATCCAGAGAGTTGAGCCTT
GCACAACTTGTATAACCAAATGTGAGCCTCACGGATTGGTTGTCCGAT
CAACAGGTTTCAAGATATCATCTGCAGTTGCTTGTGCTAGCGGAGTTT
GCGTTACAGGATCGCAGAGCCCTTCTACCGAGATTACACTCAAGTATC
CAGGGATATCCCAGTCCTCTGGGGGGGACATAGGGGTTCACATGGCA
CATGATGATCAGTCAGTTAGCTCCAAAATAGTAGCTCACTGCCCTCCC
CAGGATCCATGCCTAGTGCATGGCTGCATAGTGTGTGCTCATGGCCT
GATAAATTACCAGTGTCACACTGCTCTCAGTGCCTTTGTTGTTGTGTT
CGTATTTAGCTCTGTCGCAATAATTTGTTTGGCCATTCTTTATAAAGTT
CTCAAGTGCCTAAAGATTGCCCCAAGGAAAGTTCTGGATCCACTAATG
TGGATTACTGTTTTCATCAGATGGGTGTATAAGAAGATGGTTGCCAGA
GTAGCAGACAATATCAATCAGGTGAACAGGGAAATAGGATGGATGGA
AGGAGGCCAGCTGGCTCTAGGGAACCCTGCCCCTATTCCTCGTCATG
CTCCAATTCCACGTTATAGCACATACCTAATGCTACTATTGATTGTCT
CATATGCATCAGCATGTTCAGAACTGATTCAGGCAAGCTCCAGAATCA
CCACTTGCTCCACAGAAGGTGTCAACACCAAGTGTAGGCTGTCTGGC
ACAGCATTAATCAGGGCAGGGTCAGTTGGGGCAGAGGCTTGTTTGAT
GTTAAAGGGGGTCAAGGAAGACCAAACCAAGTTTTTGAAGATAAAAA
CTGTCTCAAGTGAGCTATCGTGCAGGGAGGGCCAGAGCTATTGGACT
GGGTCCTTTAGCCCTAAATGTCTGAGCTCAAGGAGATGCCATCTTGTC
GGGGAATGTCATGTGAATAGGTGTCTGTCTTGGAGAGACAATGAAAC
CTCAGCAGAATTTTCATTTGTTGGGGAAAGCACGACCATGCGGGAGA
ACAAGTGTTTTGAGCAGTGTGGAGGATGGGGATGTGGGTGTTTCAAT
GTGAACCCATCTTGCTTATTTGTGCACACGTATCTGCAGTCAGTCAGA
AAAGAGGCCCTTAGAGTTTTCAACTGTATCGATTGGGTGCATAAACTC
ACTCTAGAGATTACTGACTTTGATGGCTCTGTTTCAACAATAGACCTG
GGAGCATCATCTAGCCGTTTCACAAACTGGGGTTCAGTTAGCCTCTCA
CTGGACGCAGAGGGCATTTCAGGCTCAAACAGCTTTTCCTTCATTGAG
AGCCCAGGCAAAGGGTATGCAATTGTTGATGAGCCATTCTCAGAAAT
TCCTCGGCAAGGGTTCTTGGGGGAGATCAGGTGCAATTCAGAATCTT
CAGTCCTGAGTGCTCATGAATCATGCCTTAGGGCACCAAATCTTATTT
CATACAAGCCCATGATAGATCAGTTGGAGTGCACAACAAATCTGATTG
ATCCCTTTGTTGTCTTTGAGAGGGGCTCTCTGCCACAGACAAGGAATG
ACAAAACCTTTGCAGCTTCAAAAGGAAATAGGGGTGTTCAAGCTTTCT
CTAAGGGCTCTGTACAGGCTGATCTAACACTGATGTTTGACAATTTTG
AGGTGGACTTTGTGGGAGCAGCCGTGTCTTGTGATGCCGCCTTCTTA
AATTTGACAGGTTGCTATTCCTGCAATGCAGGGGCCAGAGTCTGCCT
GTCTATCACATCCACAGGAACTGGAACTCTCTCTGCCCACAATAAAGA
TGGATCTCTGCATATAGTTCTTCCATCAGAGAATGGAACAAAAGATCA
GTGTCAGATACTACACTTCACTGTACCTGAGGTAGAGGAGGAGTTTAT
GTACTCTTGTGATGGAGATGAGCGGCCTCTGTTGGTGAAGGGAACCC
TGATAGCTATTGATCCATTTGATGATAGGCGAGAAGCAGGGGGGGAA
TCAACAGTTGTGAATCCAAAATCTGGATCTTGGAATTTCTTTGACTGG
TTTTCTGGACTCATGAGTTGGTTTGGAGGGCCTCTTAAGACTATACTC
CTCATTTGCCTGTATGTAGCATTATCAATTGGGCTCTTTTTCCTTCTTA
TATATCTTGGAAGAACAGGCCTCTCTAAAATGTGGCTTGCTGCCACCA
AGAAAGCCTCATAG

The sequence starting from the fourth methionine codon of the RVFV M segment was codon-optimized for expression in mammalian cells by the GenScript cooperation (Piscataway, USA). The codon optimized sequence is as follows:

ATGGCCGGAATCGCCATGACAGTGTTGCCTGCACTGGCCGTGTTTGC
TTTGGCTCCCGTGGTGTTTGCTGAAGACCCGCACCTGCGCAACCGTC
CTGGCAAGGGCCACAACTATATTGACGGCATGACCCAGGAAGACGCT
ACATGTAAGCCGGTGACATATGCTGGCGCCTGCTCTAGCTTCGACGT
GCTCCTGGAAAAGGGAAAATTCCCACTGTTTCAGTCCTATGCTCATCA
CCGCACCCTGCTGGAGGCCGTCCACGACACAATTATCGCAAAGGCCG
ATCCCCCTAGCTGCGACCTGCAGAGCGCCCATGGCAACCCGTGCATG
AAAGAGAAACTGGTGATGAAAACACATTGCCCGAATGACTACCAGTC
TGCACACTATCTCAACAATGACGGCAAGATGGCTTCCGTGAAATGCC
CACCAAAGTACGAACTGACCGAGGATTGTAACTTTTGCCGCCAGATG
ACGGGCGCAAGTCTTAAGAAGGGTAGTTACCCTCTGCAGGACCTGTT
TTGTCAGTCCTCAGAGGACGACGGCAGCAAGCTCAAAACTAAAATGA
AGGGCGTGTGCGAGGTGGGTGTGCAAGCCCTTAAAAAGTGCGACGGC
CAGCTCTCCACCGCCCACGAAGTGGTCCCTTTTGCTGTTTTTAAGAAT
AGCAAGAAGGTGTACCTCGACAAACTGGATCTGAAAACTGAAGAAAA
CCTGCTTCCTGATAGTTTCGTGTGCTTCGAGCACAAAGGCCAGTACAA
GGGTACCATGGACTCCGGTCAGACCAAACGCGAGCTGAAATCCTTCG
ACATTTCCCAGTGCCCCAAGATCGGAGGACACGGAAGCAAGAAATGC
ACCGGCGACGCCGCCTTCTGTAGCGCCTACGAATGCACTGCCCAATA
TGCCAACGCTTATTGCTCTCACGCCAACGGTTCTGGCGTGGTGCAGA
TTCAGGTGTCCGGCGTCTGGAAGAAGCCGTTGTGTGTGGGCTATGAA
CGCGTGGTGGTGAAGCGGGAGTTGAGCGCTAAGCCCATCCAGCGTGT
GGAGCCATGCACCACCTGCATCACAAAGTGTGAACCACACGGTCTGG
TGGTGAGGTCTACCGGATTTAAGATTAGCTCTGCAGTCGCCTGTGCA
AGTGGCGTGTGTGTCACTGGCTCACAGAGTCCCTCAACGGAAATCAC
TTTGAAGTATCCCGGCATCAGCCAAAGCTCTGGAGGCGATATCGGCG
TCCATATGGCCCACGACGACCAGAGCGTGAGCTCAAAGATTGTTGCC
CACTGCCCCCCGCAGGACCCTTGCCTTGTGCACGGCTGCATTGTGTG
CGCCCACGGATTGATTAACTACCAATGCCACACCGCACTCAGCGCCTT
TGTCGTGGTTTTTGTGTTTTCTTCCGTTGCAATCATTTGCCTGGCCAT
CCTGTACAAAGTCCTCAAATGCCTGAAAATTGCCCCTAGGAAGGTCCT
CGACCCGTTGATGTGGATTACGGTGTTCATCCGATGGGTGTATAAGA
AGATGGTGGCAAGGGTGGCAGATAACATTAACCAGGTGAACAGAGAG
ATAGGATGGATGGAAGGTGGCCAGTTGGCACTTGGTAACCCTGCCCC
CATCCCTCGACACGCCCCCATTCCGAGATATAGCACCTACCTCATGCT
GCTTCTGATCGTGAGCTACGCATCCGCCTGCAGCGAGCTGATTCAGG
CCAGCAGTAGAATCACGACGTGCAGTACAGAAGGAGTGAACACCAAA
TGCCGCCTGTCCGGAACCGCCCTGATTCGCGCCGGCTCCGTCGGCGC
CGAGGCCTGTCTCATGCTCAAGGGCGTGAAGGAGGACCAGACCAAAT
TCCTGAAGATCAAGACTGTTTCATCTGAACTCTCATGTCGGGAGGGAC
AGTCCTACTGGACAGGTAGCTTCAGTCCAAAGTGTCTTTCCTCCCGTC
GCTGTCACCTGGTCGGGGAATGTCATGTGAATAGGTGTCTGTCATGG
CGCGACAACGAGACTTCCGCCGAATTTTCTTTCGTGGGTGAATCCACC
ACCATGCGGGAAAATAAATGTTTCGAACAGTGCGGCGGCTGGGGTTG
TGGCTGCTTCAACGTGAACCCGTCTTGCCTCTTTGTTCATACCTATCT
GCAATCTGTGCGCAAGGAAGCTCTGCGCGTTTTTAATTGTATCGACTG
GGTGCATAAGCTCACATTGGAAATCACAGATTTTGACGGCTCCGTCA
GCACCATCGACCTGGGAGCTTCTTCATCACGATTTACAAACTGGGGTA
GCGTGAGTCTCTCCCTGGATGCCGAAGGTATTTCAGGCAGCAACAGT
TTTAGTTTCATCGAATCCCCTGGCAAGGGTTATGCCATCGTGGACGAA
CCTTTCTCCGAGATCCCAAGGCAGGGCTTCCTTGGAGAGATCAGGTG
CAACTCAGAAAGCTCCGTGTTGAGTGCTCATGAGAGTTGTCTGAGGG
CCCCGAACCTGATCTCCTATAAGCCCATGATTGACCAGCTTGAGTGCA
CAACAAATCTTATAGATCCCTTCGTCGTGTTTGAAAGAGGCTCCCTCC
CCCAGACCCGCAACGACAAGACGTTCGCAGCTTCTAAGGGCAACCGT
GGAGTCCAGGCCTTTAGCAAGGGTTCCGTGCAGGCCGACCTGACATT
GATGTTCGATAACTTCGAGGTGGATTTCGTCGGAGCCGCTGTCTCCT
GCGATGCAGCATTTCTGAATCTGACTGGCTGCTATAGTTGCAATGCTG
GAGCACGCGTGTGCCTGAGCATTACCTCCACTGGTACAGGTACCCTG
TCCGCCCACAATAAAGATGGAAGTCTTCACATCGTGCTGCCTAGCGA
GAACGGCACAAAGGACCAATGTCAGATTCTGCACTTTACCGTGCCCG
AGGTGGAGGAAGAGTTCATGTACTCCTGTGATGGCGATGAGAGGCCT
CTGCTGGTCAAGGGCACTCTCATCGCCATTGACCCTTTTGATGACAGA
CGCGAGGCTGGCGGAGAGAGCACTGTCGTTAACCCAAAGAGCGGCTC
TTGGAATTTCTTTGACTGGTTCAGCGGACTCATGTCCTGGTTTGGAGG
CCCACTCAAGACGATTCTCCTGATCTGCCTGTACGTGGCTCTGAGTAT
CGGACTCTTCTTCCTCCTGATCTATCTCGGAAGAACCGGCTTGTCAAA
AATGTGGCTGGCCGCTACAAAGAAAGCCAGTTAA

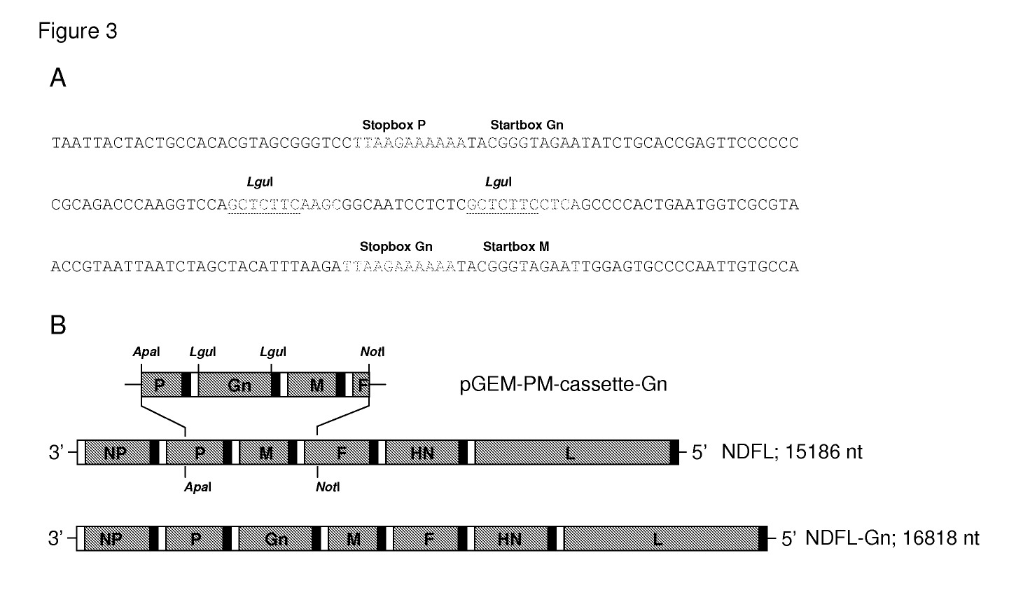

The resulting plasmid was named pUC57-GnGcOpt. The Gn gene was PCR amplified from this plasmid using the Expand High-Fidelity PCR system (Roche, Almere, The Netherlands). The PCR product was cloned into pcDNA3.1/V5-His according to the instructions of the manufacturer (Invitrogen, Breda, The Netherlands) and sequenced using an ABI PRISM 310 genetic analyzer (Applied Biosystems, Nieuwerkerk a/d IJssel, The Netherlands). The Gn gene in plasmid pcDNA3.1/V5-His-Gn is flanked by two LguI sites, which were used to transfer the gene to a plasmid named pGEM-PM-cassette (kindly provided by Olav de Leeuw, CVI-WUR, Lelystad, The Netherlands). The pGEM-PM-cassette plasmid contains the sequence that is located between unique ApaI and NotI sites in the pNDFL plasmid. The sequence between the ApaI and NotI sites in the pNDFL plasmid as well as newly introduced transcription start and stop boxes and two LguI sites that can be used for insertion of foreign genes (FIG. 3). The sequence between the ApaI and NotI sites of pNDFL was exchanged for the corresponding region of the resulting plasmid, pGEM-PM-cassette-Gn., was exchanged for the corresponding fragment of plasmid pNDFL. The resulting plasmid was named pNDFL-Gn

Rescue of recombinant viruses from cDNAs. To generate recombinant NDVs from pNDFL and pNDFL-Gn, QM-5 cells were seeded in six-well culture dishes and subsequently incubated with FPV-T7 for 1 h at 37° C. Subsequently, the cells were cotransfected with pNDFL or pNDFL-Gn (1 μg), pCIneoNP (800 ng), pCIneoP (400 ng) and pCIneoL (400 ng) using 8 μl Fugene HD according to the instructions from the manufacturer (Roche, Mannheim, Germany). After 18 to 24 h, allantoic fluid was added to a final concentration of 5%. After three to four days, the culture supernatant was harvested, passed through a 0.22 μm filter, and subsequently inoculated into the allantoic cavities of 9 to 11-day-old embryonated SPF eggs. Virus production was confirmed by standard hemagglutination assays. Viral genomic RNAs were isolated and used for reverse-transcriptase PCR. A PCR product covering the Gn gene was sequenced using an ABI PRISM 310 genetic analyzer. The Gn gene in virus NDFL-Gn remained unchanged during at least four egg passages.

Immunoperoxidase monolayer assays (IPMAs). Monolayers were washed with D-PBS (Invitrogen, Breda, the Netherlands), dried to the air, and frozen at −20° C. The monolayers were fixed with paraformaldehyde (4% w/v in PBS) for 15 min and subsequently washed with PBS. For detection of NDV, an F-protein-specific mouse monoclonal antibody (mAb 8E12A8C3) was used as the primary antibody. For detection of RVFV Gn, a sheep polyclonal antiserum was used (antiserum 841, kindly provided by Dr. Christiaan Potgieter, ARC-OVI). Antisera were diluted in His buffer (0.5M NaCl/1% Tween-80 [Genfarma, Zaandam, The Netherlands]/0.1% NaN3) containing 4% horse serum. After incubation at 37° C. for 1 h, the plates were washed three times with PBS-T. Peroxidase conjugated rabbit anti-sheep antibodies (1:2000, Abcam, Cambridge, UK), or rabbit anti-mouse antibodies (1:500, DAKO, Heverlee, Belgium) were used as the secondary antibodies. Activity of peroxidase was detected using 3-amino-9-ethyl-carbazole (Sigma, St. Louis, USA) as the substrate.

Immunofluorescence analysis (IFA). Plates containing BHK-21 cells previously infected with NDFL or NDFL-Gn were washed with PBS and incubated with 4% paraformaldehyde. Cells were washed three times with PBS and subsequently incubated for 1 h with PBS containing 4% fetal bovine serum (FBS). The polyclonal sheep antiserum 841 was used as the primary antibody (1:200 in PBS/4% FBS). FITC-conjugated rabbit anti-sheep antibodies (Santa Cruz Biotechnology, Santa Cruz, USA) were used as the secondary antibody (1:200 in PBS/4% FBS). Samples were analyzed using a Zeiss fluorescence microscope.

Inoculation of calves. Dutch Holstein Frisian or mixed breed cattle seven to nine months of age were randomly allotted into groups of three animals. All calves were inoculated with a total amount of 2.10⁷ TCID₅₀ of recombinant virus. Calves from group 1 (numbers 3451, 3452 and 3453) were inoculated in each nostril with 5 ml growth medium containing 10^{6.3} TCID₅₀ of virus, using a nozzle. Calves from group 2 (numbers 3454, 3455 and 3456) were inoculated in the neck muscle with 2 ml tissue culture medium containing 10⁷TCID₅₀/ml NDFL. Calves of group 3 (numbers 3457, 3458 and 3459) and 4 (numbers 3460, 3461 and 3462) were vaccinated with the NDFL-Gn virus via the intranasal route or the intramuscular route, respectively, as described above.

Virus inoculations were performed on days 0 and 28. Body temperatures were monitored daily after the first and second inoculation, starting from one day before the inoculation until twelve or ten days after, respectively.

The normal body temperature of calves younger than one year is between 38.5 and 39.5° C. Accordingly, fever was defined as a body temperature above 39.5° C. Serum was collected on days 0, 3, 7, 10, 14, 21, 28, 31, 35, 38 and 42. Heparin blood, nasal swabs, throat swabs and lung lavages, to be used for virus isolation, were collected on days 0, 1, 3 and 6. Eagle's MEM (4 ml) containing 2% FCS and 10% ABII was added to nasal swabs and throat swabs and incubated for 5 min. Samples were cleared by low-speed centrifugation and supernatant was stored at −70° C. Heparin blood and serum samples were stored at −70° C.

Isolation of virus from pooled samples of heparin blood, nasal swabs, throat swabs or lung lavages was performed by inoculation of 9-11 day old embryonated hens eggs. Virus production was confirmed by a standard hemagglutination assay.

These experiments were approved by the Ethics Committee for Animal Experiments of the Central Veterinary Institute of Wageningen UR.

Virus neutralization tests. Virus neutralization tests (VNTs) with RVFV strain M35/74 were performed in biosafety class III glove boxes in the BSL-3 laboratory. Sera collected three weeks after the second vaccination were individually tested in quadruplet. The serum pools were diluted in 100 μl CO₂-independent medium (GIBCO), supplemented with 1% penicillin/streptavidin (GIBCO), L-glutamine 2 mM (GIBCO) and 5% FCS. Two-fold serial dilutions of the sera (50 μl) were mixed in 96-well plates with 50 μl of culture medium containing ˜100 TCID50 of RVFV. After 2.5 h incubation at RT, 50 μl culture medium containing 4×10⁴ BHK-21 cells was added to each well. After a 3-4 day incubation period at 37° C., the cultures were scored for cytopathic effect. Titres were calculated using the Spearman-Karber method.

ELISA. The NDV-specific IgG response was determined using the IDEXX NDV antibody test kit (IDEXX, Maine, USA), which was modified for analysis of cow antibodies. Plates were incubated for 15 min with blocking buffer (PBS containing 2.9% w/v NaCl, 0.5% v/v Tween-80 [Genfarma, Zaandam], 10% w/v skim milk [Difco], 10% w/v BSA fraction V, 1% v/v Tergitol NP-9 [Sigma-Aldrich, St. Louis, USA]). Plates were incubated with sera diluted 1:20 in blocking buffer for 30 min at RT and subsequently washed 2×3 times with distilled water containing 0.05% Tween-80. Peroxidase-conjugated rabbit anti-cow antibodies (DakoCytomation, Glostrup, Denmark), diluted 1:5000 in PBS containing 2% w/v NaCl and 0.5% v/v Tween-80 (Genfarma, Zaandam), were used as secondary antibodies. Incubation was performed for 30 min at RT, and plates were subsequently washed. After staining using a standard 3,3′, 5,5′-tetramethylbenzidine substrate solution, the optical density (O.D.) was measured at 450 nm.

### Results

Construction and characterization of NDFL-Gn. NDV uses a single promoter for the transcription of its genes. The RVFV Gn gene, present in a new transcription cassette, was introduced into the DNA copy of NDV strain LaSota (i.e. NDFL). With the aim to attain high production levels, the gene was inserted between the coding sequences for the P and M proteins (FIG. 3). The insert site between P and M was selected to retain the normal ratio of the NP and P proteins, which are both important for effective genome replication.

Rescue of NDV recombinants was performed essentially as described previously (Peeters et al., 1999. J Virol 73: 5001-5009) Both pNDFL and pNDFL-Gn were readily recovered from inoculated embryonated hen's eggs, achieving peak titers of 10¹¹ and 10⁹ TCID₅₀/ml, respectively, in QM-5 cells. Virus titers were determined by IPMAs using either the anti-F mAb 8E12A8C3 or the polyclonal anti-RVFV sheep serum 841. Sequencing demonstrated that the Gn gene in virus NDFL-Gn remained unchanged during at least four egg passages.

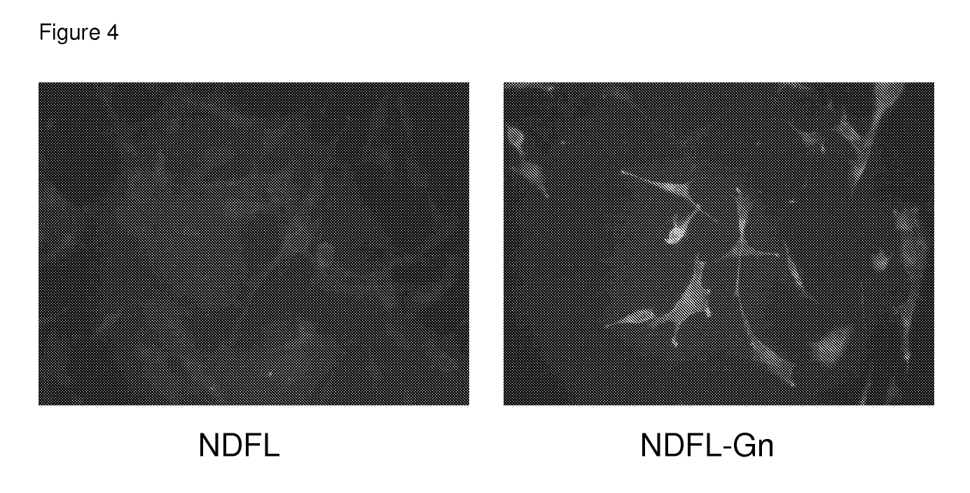

To determine if mammalian cells would enable expression of RVFV Gn from the NDV genome, BHK-21 cells were infected with the NDFL-Gn virus and expression of the Gn protein was detected by staining NDV-Gn-infected BHK-21 monolayers with the RVFV 841 antiserum (FIG. 4).

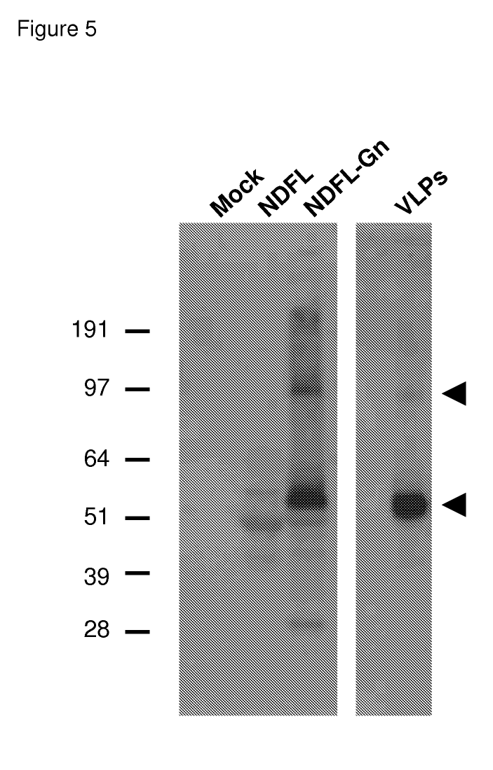

Expression of Gn from the NDV genome results in the localization of the protein at the plasma membrane (FIG. 4). NDV acquires its envelope from the plasma membrane, and is known to incorporate foreign proteins that are located at this position (Bukreyev et al., 2005; DiNapoli et al., 2007a; DiNapoli et al., 2007b). It is possible that Gn is incorporated in the NDV particle. If this is the case, its presence in allantoic fluid could stimulate the induction of a potent immune response against this protein. To investigate this possibility, allantoic fluid was placed on top of a 20% sucrose cushion and centrifuged at 80 000×g for 2 h. As a reference, previously prepared culture medium of Schneider 2 (S2) cells producing RVFV virus-like particles (VLPs) was taken along as a control. The proteins present in the resulting pellets were analyzed by Western blotting. Indeed, the Gn protein was detected in these pellets (FIG. 5). It is interesting to note that both samples seemed to contain an oligomer of the Gn protein, and that the size of Gn is somewhat larger when produced in hen's eggs when compared to Gn produced in S2 cells. This is most likely explained by differences in glycosylation of the Gn protein.

Vaccination of calves. The immunogenicity of NDFL and NDFL-Gn in calves was investigated. Animals were vaccinated via either the intranasal or the intramuscular route. After the first inoculation, one calf of each group showed hyperthermia for one or two days. One of the calves inoculated with NDFL via the intranasal route showed hyperthermia on day 26 after inoculation. One calf that was inoculated with NDFL via the intramuscular route showed hyperthermia on day 6 after inoculation (39.6° C.). One calf of the group that was inoculated via the intranasal route with NDFL-Gn showed hyperthermia at 3 days after inoculation (40.0° C.), and one calf that was inoculated via the intramuscular route with NDFL-Gn showed hyperthermia on 3 and 7 days after inoculation (39.8° C. and 39.9° C., respectively). After the second inoculation no hyperthermia was observed. In one calf that was inoculated via the intramuscular route with NDFL, nasal discharge was noted on the second day after the first inoculation and on days 18, 19 and 20 after the second inoculation. This calf did not show hyperthermia. At 19 days after the second inoculation, one calf inoculated with NDFL-Gn via the intranasal route showed nasal discharge for one day. In two calves inoculated with NDFL-Gn, diarrhoea was observed for one day. This was observed in one calf at 8 days after the first intranasal inoculation, and in one calf at 4 days after the second intramuscular inoculation. Diarrhoea did not coincide with hyperthermia.

To study if NDFL and NDFL-Gn were capable of spread in the inoculated animals, heparin blood samples, nasal swabs, throat swabs and lung lavages, collected on days 0, 1, 3 and 6, were used for virus isolation by inoculation of embryonated hens eggs. No virus was isolated from any of these samples.

The observed nasal discharge and diarrhoea did not coincide with hyperthermia and is, therefore, unlikely to result from the NDV infection. This is supported by the fact that no NDV virus could be isolated from the calves. Nasal discharge and diarrhoea are not uncommon in calves this age. The hyperthermia, although only short lived, could have resulted from the inoculation. This observation was however only in a few calves and therefore not very consistent. We conclude from these findings that NDFL and NDFL-Gn are largely, if not completely, innocuous in calves.

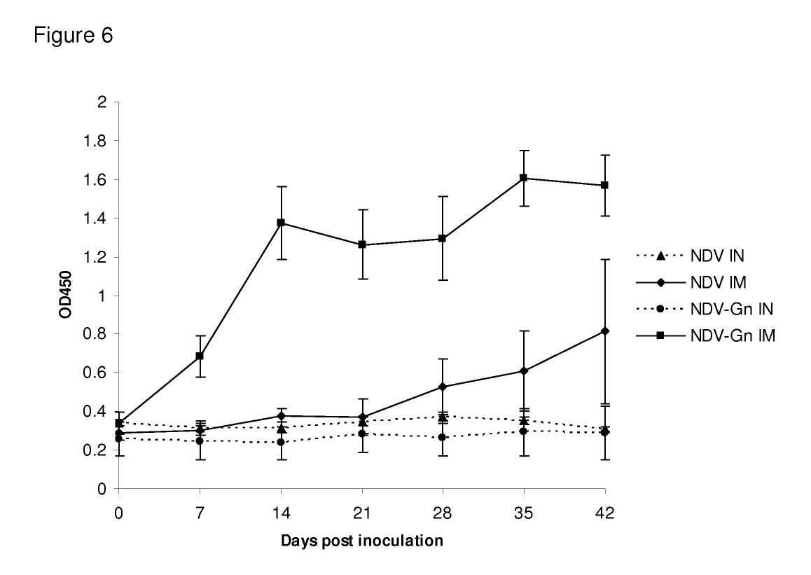

Antibody responses. To study the antibody responses elicited by NDFL and NDFL-Gn after either intranasal or intramuscular vaccination, sera were collected weekly and analyzed by a modified IDEXX NDV ELISA. Whereas inoculation via the intranasal route elicited no detectable NDV response, inoculation via the intramuscular route did induce an antibody response. Remarkably, the NDFL-Gn virus induced higher NDV-specific antibody levels when compared to NDFL (FIG. 6).

To determine if antibodies against the Gn protein were elicited by NDFL-Gn, sera obtained three weeks after the second vaccination were analyzed by IPMAs using cells that were previously transfected with plasmid pCAGGS-GnGc from which the Gn gene is expressed. Only the sera obtained from the three calves that were inoculated via the intramuscular route with the NDFL-Gn virus stained these cells (Table 2). In accordance with this result, virus neutralization assays demonstrated that only the aforementioned sera were capable of neutralizing the RVFV in vitro. The virus titres varied between 8 and 32 (Table 2).

TABLE 2
Analysis of sera^{a} from vaccinated calves

| | Calf no. | IPMA-NDV^{b} | IPMA-Gn^{c} | VNT^{d} |
|---|---|---|---|---|
| NDFL | 3451 | − | − | <2 |
| Intranasal | 3452 | − | − | <2 |
| | 3453 | − | − | <2 |
| NDFL | 3454 | + | − | <2 |
| Intramuscular | 3455 | − | − | <2 |
| | 3456 | + | − | <2 |
| NDFL-Gn | 3457 | − | − | <2 |
| Intranasal | 3458 | − | − | <2 |
| | 3459 | − | − | <2 |
| NDFL-Gn | 3460 | + | +/− | 8 |
| Intramuscular | 3461 | + | + | 32 |
| | 3462 | + | + | 16 |

^{a}Sera were obtained three weeks after the second vaccination.
^{b}The presence of antibodies against NDV was determined by staining NDFL-infected BHK-21 cells.
^{c}The presence of antibodies against Gn was determined by staining BHK-21 cells expressing the Gn gene from plasmid pCAGGS-GnGc.
^{d}VNT titers are depicted as the reciprocal value of the highest neutralizing serum dilution.

### Example 3

### Materials and Methods

Cells, plasmids and viruses. Quail fibrosarcoma cells (QM-5) were grown in Ford Dodge QT35 medium (Invitrogen, Breda, The Netherlands) containing 5% fetal calf serum (FCS) and 1% antibiotic/antimycotic (Invitrogen). BHK-21 cells were grown in GMEM containing 4% tryptose phosphate broth (Invitrogen), 1% non-essential amino acids (Invitrogen) and 10% FCS.

The cDNA clone of NDV strain LaSota, named pNDFL and the helper plasmids pCIneo-NP, pCIneo-P and pCIneo-L, have been described previously [Peeters et al. 1999. J Virol 73: 5001-9]. Plasmid pCAGGS-GnGc contains a codon-optimized GnGc gene of RVFV strain M35/74 under chicken-actin promoter control (de Boer et al., submitted for publication).

The fowlpox recombinant virus fpEFLT7pol (hereafter called FPV-T7) [Britton et al. 1996. J Gen Virol 77: 963-972] was provided by Olav de Leeuw (Central Veterinary Institute of Wageningen UR [CVI-WUR], Lelystad, The Netherlands). RVFV strain M35/74 was kindly provided by Prof. dr. Janusz Paweska (National Institute for Communicable Diseases [NICD], Johannesburg, South Africa) and Dr. Christiaan Potgieter (Agricultural Research Council-Onderstepoort Veterinary Institute [ARC-OVI], Onderstepoort, South Africa).

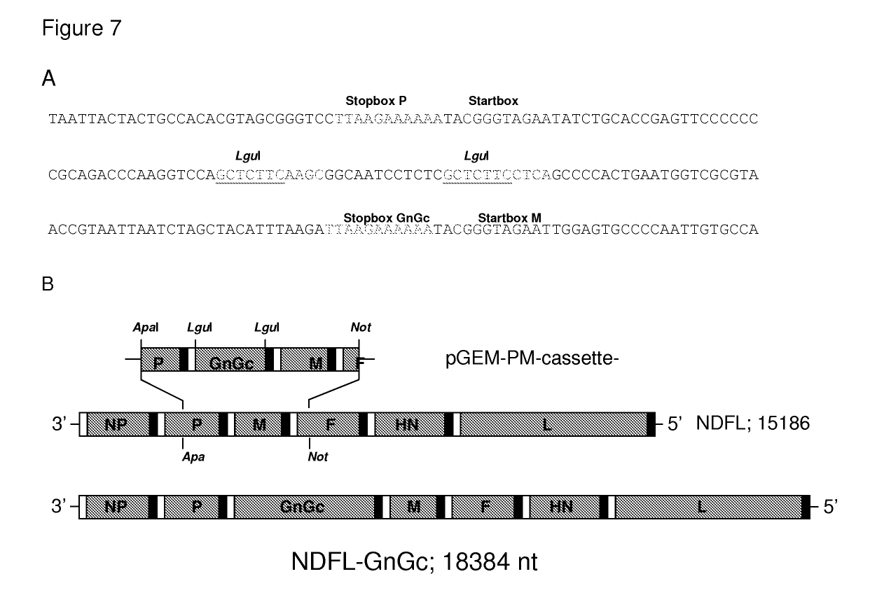

Production of NDFL-GnGc. The sequence of the M genome segment of RVFV strain M35/74 was kindly provided by Dr. Christiaan Potgieter (ARC-OVI). A synthetic DNA sequence starting from the fourth methionine codon of the RVFV M segment was synthesized and codon-optimized for expression in mammalian cells by the GenScript cooperation (Piscataway, USA). For cloning purposes, two LguI sites, flanking the GnGc gene were introduced and the gene was cloned in pUC57 by the GenScript cooperation, resulting in plasmid pUC57-GnGcOpt. The GnGc gene was sequenced using an ABI PRISM 310 genetic analyzer (Applied Biosystems, Nieuwerkerk a/d IJssel, The Netherlands). The LguI sites were used to transfer the gene to a plasmid named pGEM-PM-cassette (kindly provided by Olav de Leeuw, CVI-WUR, Lelystad, The Netherlands). The pGEM-PM-cassette plasmid contains the sequence that is located between unique ApaI and NotI sites in the pNDFL plasmid, as well as newly introduced NDV transcription start and stop boxes and two LguI sites to facilitate insertion of foreign genes (FIG. 7). The sequence between the ApaI and NotI sites of pNDFL was exchanged for the corresponding fragment of plasmid, pGEM-PM-cassette-GnGc. The resulting plasmid, pNDFL-GnGc (FIG. 7), was designed in such a way, that the DNA copy of the NDV genome complies to the rule of six [Peeters et al. 2000. Arch Virol 145: 1829-45; Calain et al. 1993. J Virol 67: 4822-30]. Recombinant virus was generated from plasmid pNDFL-GnGc using methods used for the rescue of NDFL-Gn, described in Example 2. Virus titers were determined as tissue culture 50% infective dose (TCID50) on QM-5 cells.

Characterization of NDFL-GnGc. Immunoperoxidase monolayer assays (IPMA) and immunofluorescence assays (IFA) were performed as described in Example 2. For Western blot analysis of Gn and Gc, rabbit polyclonal antibodies were used that were previously raised against a Gn-derived peptide (residues 374-CFEHKGQYKGTMDSGQTKRE-393) or a Gc-derived peptide (residues 975-VFERGSLPQTRNDKTFAASK-994) [Filone et al. 2006. Virology 356: 155-64] (de Boer et al., submitted for publication). Proteins were separated in 4 to 12% Bis-Tris gradient gels (NuPAGE, Invitrogen) and subsequently transferred to nitrocellulose membranes (Protran, Schleicher and Schuell, VWR, Amsterdam, The Netherlands). After 1 h incubation in blocking buffer (PBS/0.05% Tween-20/1% Skim milk [Difco, Becton, Dickinson and Company, Sparks, Md., USA]), the blots were incubated for 1 h with rabbit polyclonal anti-peptide antibodies, diluted in blocking buffer. Goat anti-rabbit horseradish peroxidase-conjugate (DAKO) was used as the secondary antibody and peroxidase activity was detected using the Amersham ECLTM Western blotting detection reagents (GE Healthcare, Diegem, Belgium). Vaccination and challenge of mice. Female BALB/c mice (Charles River laboratories, Maastricht, The Netherlands) were housed in groups of five animals in type III filter-top cages and kept under BSL-3 conditions. The light regime was set at 14 h light/10 h dark, the temperature at 22° C. and the relative humidity at 55%. Food and water was provided ad libitum. Groups of ten 7-week-old mice were vaccinated via the intramuscular route on days 0 and 21 with 107 TCID50 NDFL or NDFL-GnGc, present in 50 μl culture medium. One group of ten mice was left untreated (non-vaccinated). The body weights of the mice were monitored weekly and blood samples, to be used for serological tests, were obtained from the tail vein at different time points. On day 42, all mice were challenged via the intraperitoneal route with 102.7 TCID50 of RVFV strain M35/74 in 0.5 ml culture medium. The lethal challenge dose was determined after two dose titration studies (Antonis A F et al., manuscript in preparation). Challenged mice were monitored daily for visual signs of illness and mortality. At day 62 post initial immunization, all animals that survived the RVFV challenge were bled via orbital puncture under general anaesthesia using xylazine (7 mg/kg) and ketamine (70 mg/kg) and euthanized by cervical dislocation. To confirm productive infection in surviving mice, sera were analyzed for the presence of antibodies against the nucleoprotein using a modified recN ELISA (BDSL, Ayrshire Scotland, UK) and livers were tested for the presence of viral RNA by quantitative real-time reverse-transcriptase PCR using a LightCycler instrument (Roche Applied Science) as described [Drosten et al. 2002. J Clin Microbiol 40: 2323-30].

A commercially available RVFV ELISA was used to detect antibodies directed against the nucleocapsid (N) protein. This so-called recN ELISA was originally developed for analysis of sera from livestock [Paweska et al. 2008. Vet Microbiol 127: 21-8]. For analysis of the mouse sera, the ELISA was performed essentially according to the manufacturer's instructions (BDSL, Ayrshire Scotland, UK), but with the following modifications. Plates were coated with stock antigen, diluted 1:3000 and all mouse sera were analyzed in duplicate. As the secondary antibody, a peroxidase-conjugated rabbit anti-mouse antibody (DAKO, Glostrup, Denmark) was used. The cut-off was set as described [Paweska et al. 2008. Vet Microbiol 127: 21-8] at the mean value obtained from the negative control serum plus two times the corresponding standard deviation. All data were calculated relative to the controls percent positive value (PP value).

### Results

Construction and characterization of NDFL-GnGc. NDFL-GnGc was readily recovered from 9-11 day-old embryonated hens' eggs. Whereas the general production level of wildtype NDFL virus exceeded 1011 TCID50, the maximum titres of pNDFL-GnGc did not exceed 109 TCID50/ml. As expected, QM-5 or BHK-21 cells infected with NDFL-GnGc could be stained with antibodies directed against NDV and antibodies directed against RVFV in IPMAs and IFAs (data not shown).

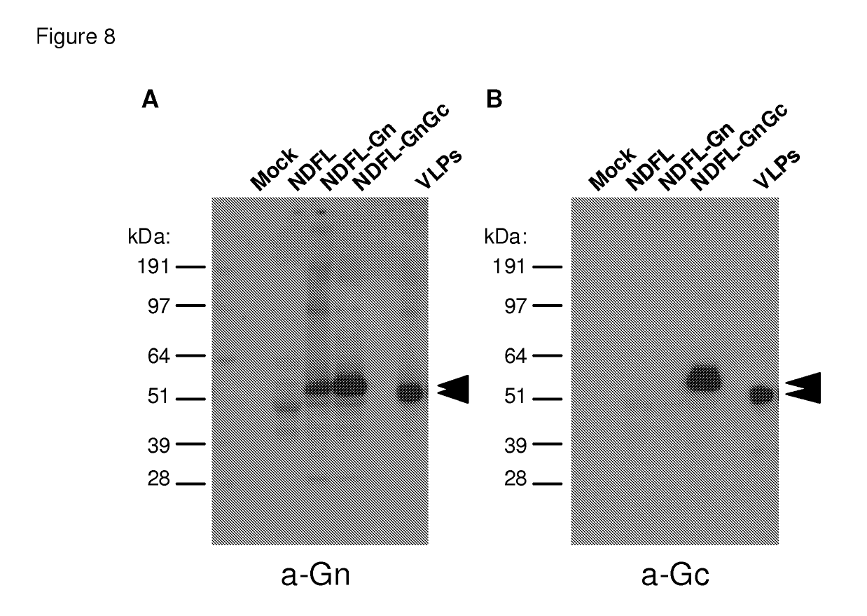

RVFV produces the glycoproteins Gn and Gc from a single protein precursor. The two glycoproteins form a heterodimer after processing of the polyprotein by host proteases in the endoplasmic reticulum [Gerrard et al. 2007. Virology 357: 124-33]. We have previously described a recombinant NDV virus that produces the RVFV Gn protein only (i.e. NDFL-Gn, Example 2). Production of Gn from the authentic precursor protein by NDFL-GnGc could result in production levels of Gn that are higher as those obtained from NDFL-Gn. Furthermore, NDFL-GnGc not only produces Gn, but also Gc, which is also known to induce neutralizing antibodies [Besselaar et al. 1992. Arch Virol 125:239-50; Besselaar et al. 1991. Arch Virol 121:111-24]. To compare the expression levels of Gn from NDFL-Gn and NDFL-GnGc, allantoic fluids containing these viruses were placed on top of a sucrose cushion and centrifuged at 80 000×g for 2 h. As a reference, previously prepared culture medium of Schneider 2 (S2) cells producing RVFV virus-like particles (VLPs, de Boer et al., submitted for publication) was taken along as a control. The proteins present in the resulting pellets were analyzed by Western blotting using Gn and Gc-specific polyclonal antibodies. As previously described (Example 2), allantoic fluid containing NDFL-Gn, contained the Gn protein (FIG. 8, panel A). The Gn protein was also detected in allantoic fluid containing NDFL-GnGc (FIG. 8, panel A). Lower exposures of the blot depicted in FIG. 8 demonstrated that the amount of Gn was considerably higher in allantoic containing NDFL-GnGc when compared to similar samples produces from NDFL-Gn (data not shown). As expected, the Gc protein was only detected in allantoic fluid containing NDFL-GnGc (FIG. 8, panel B). It is interesting to note that the molecular weight of both Gn and Gc are somewhat larger when produced in hen's eggs. This is most likely explained by differences in glycosylation of the glycoproteins when produced in bird cells or insect cells.

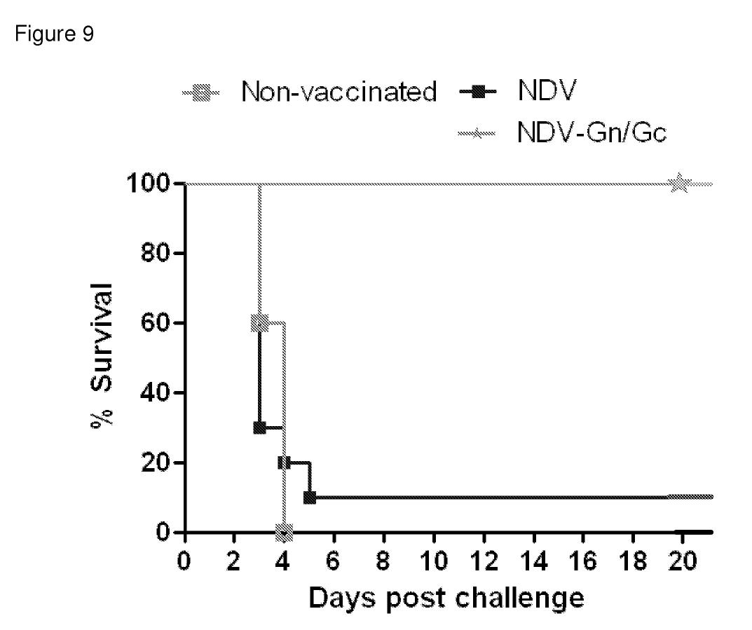

Vaccination and challenge. Groups of 10 mice were immunized via the intramuscular route with either NDFL or NDFL-GnGc and boosted three weeks later. A third group of 10 non-vaccinated mice was added as an additional challenge control group. Three weeks after the second vaccination, all mice were challenged with a known lethal dose of RVFV strain M35/74. All mice that were not inoculated succumbed to the infection within 4 days after challenge, whereas nine out of ten mice inoculated with NDV succumbed to the infection within 5 days (FIG. 9). At the end of the experiment, all these mice were positive for RVFV RNA in both the liver and the brain (data not shown). One mouse of the group inoculated with NDFL survived the RVFV challenge. Productive infection in this mouse was, however, confirmed by demonstrating N antibodies by the recN ELISA and RVFV RNA in both the liver and the brain at the end of the experiment (data not shown). All mice vaccinated with NDFL-GnGc survived the challenge, without showing any clinical signs. It is important to note that the sera of these mice, obtained at the end of the experiment, were negative in the recN ELISA. This suggested that only very limited virus replication occurred in these mice. PCR on liver and brain, however, did demonstrate viral RNA in the liver of five mice (data not shown). Our results demonstrate that NDFL-GnGc, administered via the intramuscular route, provides solid protection against a lethal RVFV challenge.

1. A method of stimulating an immune response against an antigenic protein in a mammalian subject comprising administering a composition comprising a hybrid Newcastle Disease Virus-vector (NDV-vector) comprising a nucleotide sequence encoding the antigenic protein to the subject through subcutaneous/intradermal or intramuscular administration.

2. The method of claim 1, wherein the hybrid Newcastle Disease Virus-vector (NDV-vector) is injected intramuscularly.

3. The method according to claim 1, wherein the composition further comprises an adjuvant.

4. The method according to claim 1, wherein the administration is repeated.

5. The method according to claim 1, wherein the stimulated immune response protects the subject against an infectious disease.

6. The method according to claim 1, wherein the stimulated immune response protects the subject against infection by Rift Valley fever virus, Crimean-Congo hemorrhagic fever virus, bluetongue virus, African horsesickness virus, or African swine fever virus.

7. The method according to claim 6, wherein the stimulated immune response protects the subject against infection by Rift Valley fever virus.

8. The method according to claim 6, wherein the NDV-vector is a lentogenic NDV vector.

9. The method according to claim 6, wherein the NDV-vector is NDFL or a similar infectious clone of NDV strain LaSota.

10. The method according claim 7, wherein the antigenic protein is RVFV glycoprotein Gn or Gc, or virus-like particles produced by expression of both Gn and Gc from the NDV genome.

11. The method according to claim 1, wherein the mammalian subject is selected from pets including dogs and cats; ungulates including pigs, horses, sheep, cows, and goats; and primates, including human.

12. The method according to claim 1, wherein the mammalian subject is a human.

13. A method of stimulating an immune response against an antigenic protein in a mammalian subject comprising administering a composition to the subject through parenteral administration, the composition comprising a hybrid NDV-vector comprising a nucleic acid sequence encoding the antigenic protein, wherein the mammalian subject is selected from pets including dogs and cats; ungulates including pigs, horses, sheep, cows, and goats; and primates, including human.

14. The method according to claim 11, wherein the mammalian subject is a ruminant.
